(19) United States
(12) Patent Application Publication
(10) Pub. No.: US 2006/0111324 A1
(43) Pub. Date: May 25, 2006

(54) (+)-Ttrans-isomers of (1-phosphonomethoxy-2-alkylcyclopropyl)methyl nucleoside derivatives, process for the preparation of stereoisomers thereof, and use of antiviral agents thereof

(75) Inventors: Jong-Ryoo Choi, Taejeon (KR); Jae-Taeg Hwang, Taejeon (KR); Dong-Gyu Cho, Taejeon (KR); Kee-Yoon Roh, Taejeon (KR); Chung-Hyung Kim, Taejeon (KR); Chung-Mi Kim, Taejon (KR); Min-Joon Han, Taejeon (KR); Jeong-Min Kim, Taejeon (KR); Woo-Young Cho, Taejeon (KR); Gyoung-Won Kim, Taejeon (KR); Sin-Byoung Ahn, Taejeon (KR)

Correspondence Address: BIRCH STEWART KOLASCH & BIRCH, PO BOX 747, FALLS CHURCH, VA, 22040-0747, US

(73) Assignee: LG Life Sciences, Seoul (KR)

(21) Appl. No.: 10/528,336

(22) PCT Filed: Sep. 22, 2003
(86) PCT No.: PCT/KR03/01932

§ 371 Date: Sep. 1, 2005

(30) Foreign Application Priority Data
Sep. 26, 2002 (KR) 1020020058310

(51) Int. Cl. A61K 31/675; C07F 9/6512

(52) U.S. Cl. 514/81; 544/243; 544/244; 514/86

(57) ABSTRACT

The present invention relates to (+)-trans-isomers of (1-phosphonomethoxy-2-alkylcyclopropyl)methyl nucleoside derivatives of the formula (1) which are useful as an antiviral agent (particularly, against hepatitis B virus), pharmaceutically acceptable saltss, hydrates, or solvates thereof, and processes for the preparation of stereoisomers of the compounds of the formula (1), and a composition for the treatment of viral diseases (particularly, against hepatitis B virus) comprising (+)-trans-isomer of the compound of the formula (1), pharmaceutically acceptable salt, hydrate, or solvate thereof as an active substance.

### TECHNICAL FIELD

The present invention relates to (+)-trans-isomers of (1-phosphonomethoxy-2-alkylcyclopropyl)methyl nucleoside derivatives represented by the following formula (1):

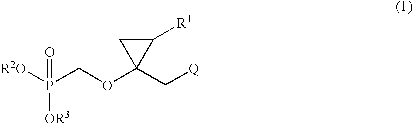

wherein,

R¹ represents C₁-C₇ alkyl,

R² and R³ independently of one another represent hydrogen, or represent C₁-C₄-alkyl optionally substituted by one or more substituents selected from a group consisting of halogen (particularly fluorine), C₁-C₄-alkoxy, phenoxy, C₇-C₁₀-phenylalkoxy, and C₂-C₅-acyloxy, or represent C₂-C₇-acyl, C₆-C₁₂-aryl, C₁-C₇-alkylaminocarbonyl, di(C₁-C₇-alkyl)aminocarbonyl) or C₃-C₆-cycloalkylaminocarbonyl, or represent —(CH₂)ₘ—OC(═O)—R⁴ wherein m denotes an integer of 1 to 12 and R⁴represents C₁-C₁₂-alkyl, C₂-C₇-alkenyl, C₁-C₅-alkoxy, C₁-C₇-alkylamino, di(C₁-C₇-alkyl)amino, C₃-C₆-cycloalkyl, or 3 to 6-membered heterocycle having 1 or 2 hetero atoms selected from a group consisting of nitrogen and oxygen,

Q represents a group having the following formulae:

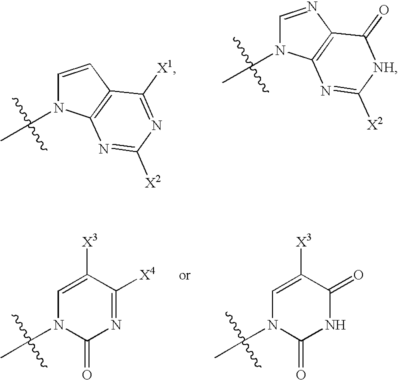

wherein,

X¹, X², X³ and X⁴ independently of one another represent hydrogen, amino, hydroxy, or halogen, or represent C₁-C₇-alkyl, C₁-C₅-alkoxy, allyl, hydroxy-C₁-C₇-alkyl, phenyl, or phenoxy, each of which is optionally substituted by nitro or C₁-C₅-alkoxy, or represent C₆-C₁₀-arylthio which is optionally substituted by nitro, amino, C₁-C₆-alkyl, or C₁-C₄-alkoxy, or represent C₆-C₁₂-arylamino, C₁-C₇-alkylamino, di(C₁-C₇-alkyl)amino, C₃-C₆-cycloalkylamino, or a structure of

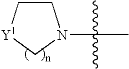

wherein n denotes an integer of 1 or 2 and Y¹ represents O, CH₂, or N—R (R represents C₁-C₇-alkyl or C₆-C₁₂-aryl), which are useful as antiviral agents (particularly, against hepatitis B virus), pharmaceutically acceptable salts, hydrates, or solvates thereof, processes for the preparation of stereoisomers thereof, and a composition for the treatment of viral disease (particularly, against hepatitis B virus) comprising (+)-trans-isomer of the compound of formula (1), pharmaceutically acceptable salt, hydrate, or solvate thereof as an active substance.

### BACKGROUND ART

Purine or pyrimidine derivatives have anti-cancer and antiviral activity, and more than 10 kinds of the compounds including AZT, 3TC, and ACV have already been commercialized. Particularly, since acyclic nucleoside phosphonate derivatives show a potent antiviral effect, cidofovir, tenofovir adefovir have been commercialized as antiviral agents, and many compounds including MCC-478 now entered into the clinical trial phases. However, the earlier developed compounds were not perfect in the aspects of toxicity or pharmaceutical activity. Thus, a compound having no toxicity as well as superior activity is still desirable. The prior researches for purine or pyrimidine derivatives or acyclic nucleoside phosphonate derivatives as reported heretofore are patents such as U.S. Pat. No. 5,817,647; U.S. Pat. No. 5,977,061; U.S. Pat. No. 5,886,179; U.S. Pat. No. 5,837,871; U.S. Pat. No. 6,069,249; WO 99/09031; WO96/09307; WO95/22330; U.S. Pat. No. 5,935,946; U.S. Pat. No. 5,877,166; and U.S. Pat. No. 5,792,756; and journals such as International Journal of Antimicrobial Agents 12 (1999), 81-95; Nature 323 (1986), 464; Heterocycles 31(1990), 1571; J. Med. Chem. 42 (1999), 2064; Pharmacology & Therapeutics 85 (2000), 251; Antiviral Chemistry & Chemotherapy 5 (1994), 57-63.; Bioorganic & Medicinal Chemistry Letters 10 (2000) 2687-2690; Biochemical Pharmacology 60 (2000), 1907-1913; Antiviral Chemistry & Chemotherapy 8 (1997) 557-564; and Antimicrobial Agent and Chemotherapy 42 (1999) 2885-2892.

Furthermore, the compounds of formula (1) have two or more asymmetric carbons, and so have four or more isomers. Isomers of the compounds having asymmetric carbons have different biological properties as well as different physiochemical properties each other. By separating and resolving those isomers, the researches for developing new medicines which are more useful to human being have been recently increased. The earlier research results for those isomers disclosed in patents such as U.S. Pat. No. 4,018,895; U.S. Pat. No. 4,194,009; U.S. Pat. No. 5,618,829; U.S. Pat. No. 5,204,446; U.S. Pat. No. 5,719,104; EP 0545425A1; and EP 0369685A1; and in journals such as Antimicrobial Agents and Chemotherapy 35 (1991)1386-1390; Antimicrobial Agents and Chemotherapy 36 (1992) 672-676; and J. Med. Chem. 31, (1988)1412-1417.

### DISCLOSURE OF THE INVENTION

The present inventors have synthesized (1-phosphonomethoxy-2-alkylcyclopropyl)methyl nucleoside derivatives represented by the formula (1), and found processes for preparation of their optical isomers effectively by separating and resolving their mixtures. Also, the present inventors succeeded in discovering that among the stereoisomers of the compounds of formula (1), (+)-trans-isomers are superior to other commercialized or developing medicines in view of pharmaceutical activity as antiviral agents (particularly against hepatitis B virus), and thus completed the present invention.

Therefore, one object of the present invention is to provide (+)-trans-isomers of the compounds of formula (1), pharmaceutically acceptable salts, hydrates, or solvates thereof, which have excellent utility as antiviral agents (particularly, against hepatitis B virus).

It is another object of the present invention to provide processes for the preparation of stereoisomers of the compounds of formula (1).

It is still another object of the present invention to provide preparation processes of the compound of formula (2) that can be used as a starting material when preparing the compound of formula (1).

Also, it is still another object of the present invention to provide a composition for the treatment of viral diseases (particularly, against hepatitis B virus) comprising (+)-trans-isomer of the compound of formula (1), pharmaceutically acceptable salt, hydrate, or solvate thereof as an active substance.

### BEST MODE FOR CARRYING OUT THE INVENTION

The compound of formula (l), as represented below, is a type of (1-phosphonomethoxy-2-alkylcyclopropyl)methyl nucleoside derivative having a natural base, such as adenine, guanine, uracil, cytosine, thymine, or derivatives thereof, and having two or more asymmetric carbon atoms:

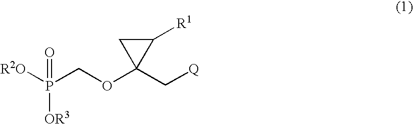

wherein,

R¹ represents C₁-C₇ alkyl,

R² and R³ independently of one another represent hydrogen, or represent C₁-C₄-alkyl optionally substituted by one or more substituents selected from a group consisting of halogen (particularly fluorine), C₁-C₄-alkoxy, phenoxy, C₇-C₁₀-phenylalkoxy, and C₂-C₅-acyloxy, or represent C₂-C₇-acyl, C₆-C₁₂-aryl, C₁-C₇-alkylaminocarbonyl, di(C₁-C₇-alkyl)aminocarbonyl or C₃-C₆-cycloalkylaminocarbonyl, or represent —(CH₂)ₘ—OC(═O)—R⁴ wherein m denotes an integer of 1 to 12 and R⁴represents C₁-C₁₂--alkyl, C₂-C₇-alkenyl, C₁-C₅-alkoxy, C₁-C₇-alkylamino, di(C₁-C₇-alkyl)amino, C₃-C₆-cycloalkyl, or 3 to 6-membered heterocycle having 1 or 2 hetero atoms selected from a group consisting of nitrogen and oxygen,

Q represents a group having the following formulae:

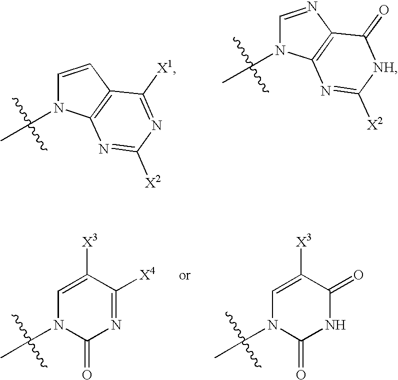

wherein,

X¹, X^{2,} X³ and X⁴ independently of one another represent hydrogen, amino, hydroxy, or halogen, or represent C₁-C₇-alkyl, C₁-C₅-alkoxy, allyl, hydroxy-C₁-C₇-alkyl, phenyl, or phenoxy, each of which is optionally substituted by nitro or C₁-C₅-alkoxy, or represent C₆-C₁₀-arylthio which is optionally substituted by nitro, amino, C₁-C₆-alkyl, or C₁-C₄-alkoxy, or represent C₆-C₁₂-arylamino, C₁-C₇-alkylamino, di(C₁-C₇-alkyl)amino, C₃-C₆-cycloalkylamino, or a structure of 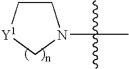
wherein n denotes an integer of 1 or 2 and Y¹ represents O, CH₂, or N—R (R represents C₁-C₇-alkyl or C₆-C₁₂-aryl).

Also, the compound according to the present invention can form a pharmaceutically acceptable salt. Such salt includes non-toxic acid addition salt containing pharmaceutically acceptable anion, for example salt with inorganic acids such as hydrochloric acid, sulfuric acid, nitric acid, phosphoric acid, hydrobromic acid, hydriodic acid, etc.; salt with organic carboxylic acids such as tartaric acid, formic acid, citric acid, acetic acid, trichloroacetic acid, trifluoroacetic acid, gluconic acid, benzoic acid, lactic acid, fumaric acid, maleic acid, etc.; or salt with sulfonic acids such as methanesulfonic acid, benzenesulfonic acid, p-toluenesulfonic acid, naphthalenesulfonic acid, etc., but preferably with sulfuric acid, methanesulfonic acid, hydrohalic acid, etc.

As (+)-trans-isomer of the compound of formula (1) showing potent pharmaceutical activity, preferred compounds are those wherein R¹ represents C₁-C₃ alkyl, R² and R³ independently of one another represent hydrogen, or represent C₁-C₄-alkyl optionally substituted by one or more substituents selected from a group consisting of fluorine, C₁-C₄-alkoxy and phenoxy, or represent —(CH₂)ₘ—OC(═O)—R⁴ wherein m denotes an integer of 1 to 12, and R⁴ represents C₁-C₅-alkyl or C₁-C₅-alkoxy, Q represents

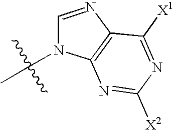

wherein, X¹ represents hydrogen, hydroxy, amino, or 4-methoxyphenylthio, and X² represents hydrogen or amino.

The typical examples for (+)-trans-isomer of the compound of formula (1) according to the present invention are described in the following Table 1.

TABLE 1a

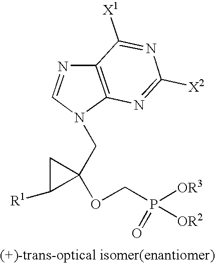

| COM. NO. | R¹ | R² & R³ | X¹ | X² |
|---|---|---|---|---|
| 1 | CH₃ | H | OH | NH₂ |
| 2 | CH₃ | H | H | NH₂ |
| 3 | CH₃ | H | NH₂ | H |
| 4 | CH₃ | H | 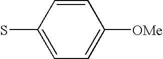 | NH₂ |
| 5 | CH₃ | H | Cl | NH₂ |
| 6 | CH₃ | 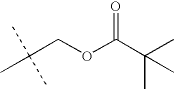 | H | NH₂ |
| 7 | CH₃ | 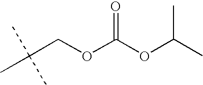 | H | NH₂ |
| 8 | CH₃ | 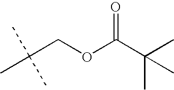 | 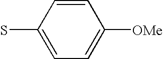 | NH₂ |
| 9 | CH₃ | 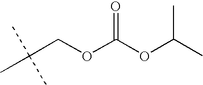 | 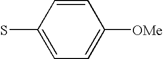 | NH₂ |
| 10 | CH₃ | 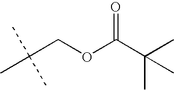 | NH₂ | H |
| 11 | CH₃ | 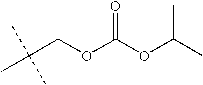 | NH₂ | H |
| 12 | C₂H₅ | H | OH | NH₂ |
| 13 | C₂H₅ | H | H | NH₂ |
| 14 | C₂H₅ | H | NH₂ | H |
| 15 | C₂H₅ | H | 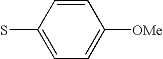 | NH₂ |

TABLE 1b

| | | | | |
|---|---|---|---|---|
| 16 | C₂H₅ | H | Cl | NH₂ |
| 17 | C₂H₅ | 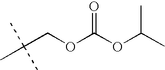 | H | NH₂ |
| 18 | C₂H₅ | 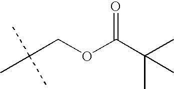 | H | NH₂ |
| 19 | C₂H₅ | 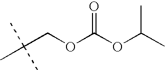 | NH₂ | H |
| 20 | C₂H₅ | 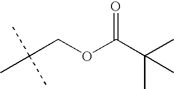 | NH₂ | H |
| 21 | C₂H₅ | 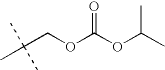 | 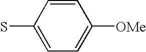 | NH₂ |
| 22 | C₂H₅ | 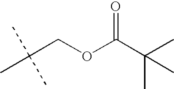 | 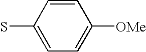 | NH₂ |
| 23 | C₃H₇ | H | OH | NH₂ |
| 24 | C₃H₇ | H | H | NH₂ |
| 25 | C₃H₇ | H | Cl | NH₂ |
| 26 | C₃H₇ | H | NH₂ | H |
| 27 | C₃H₇ | H | 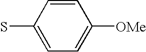 | NH₂ |
| 28 | C₃H₇ | 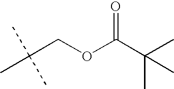 | H | NH₂ |
| 29 | C₃H₇ | 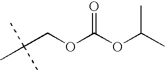 | H | NH₂ |
| 30 | C₃H₇ | 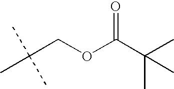 | NH₂ | H |
| 31 | C₃H₇ | 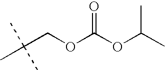 | NH₂ | H |
| 32 | C₃H₇ | 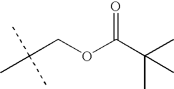 | 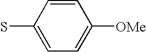 | H |
| 33 | C₃H₇ | 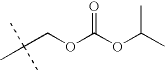 | 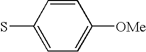 | H |
| 34 | CH₃ | iso-propyl | Cl | NH₂ |
| 35 | C₂H₅ | iso-propyl | Cl | NH₂ |

The present inventors found that absolute configuration of (+)-trans-isomer of the compound of formula (1) according to the present invention is (1S,2S).

The compound of formula (1), which is useful as antiviral agents, can be prepared by the following processes.

The preparation processes of the compound of formula (1) can be characterized in that,

(a) a compound represented by the following formula (2):

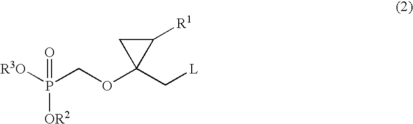

in which R¹, R² and R³ are defined as previously described, and L represents a leaving group, preferably methanesulfonyloxy, p-toluenesulfonyloxy, or halogen, is reacted with a compound represented by the following formula (3):
QH (3)

in which Q is defined as previously described, to produce the compound of formula (1), or

(b) a compound represented by the following formula (4):

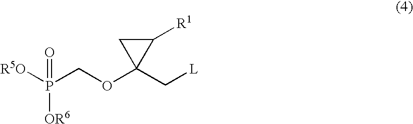

in which R¹ and L are defined as previously described, and R⁵ and R⁶ independently of one another represent C₁-C₇ alkyl, is reacted with the compound of formula (3) to produce a compound represented by the following formula (5):

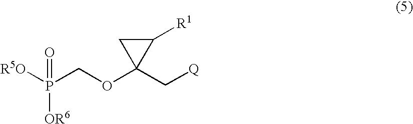

in which R¹, R⁵, R⁶ and Q are defined as previously described, and the resulting compound of formula (5) is hydrolyzed in the presence of a Lewis acid to produce a compound represented by the following formula (1a):

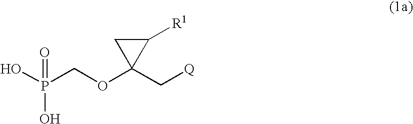

in which R¹ and Q are defined as previously described, or

(c) groups R^{2′} and R^{3′} are introduced into the compound of formula (1a) to produce a compound represented by the following formula (1b):

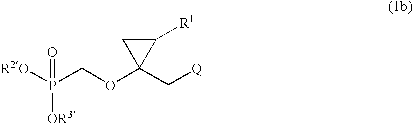

in which R¹ and Q are defined as previously described, and R^{2′} and R^{3′} represent R² and R³ with the exception of hydrogen, respectively, or further the compound thus obtained is subjected to conventional conversions (see: U.S. Pat. Nos. 6,037,335; 5,935,946; and 5,792,756).

In the above process variants (a) to (c) for preparing the compound of formula (1), the reactions may be carried out in a solvent and in the presence of base. As the solvent, one or more selected from a group consisting of dimethylformamide, dichloromethane, tetrahydrofuran, chloroform, 1-methyl-2-pyrrolidinone, and dimethylacetamide can be mentioned, and as the base, one or more selected from a group consisting of sodium hydride, sodium carbonate, potassium carbonate, sodium bicarbonate, potassium bicarbonate, potassium t-butoxide, hydrogen bis(trimethylsilyl)amide, sodium amide, cesium carbonate, and potassium bis(trimethylsilyl)amide can be mentioned. The Lewis acid which can be used in the process variant (b) includes trimethylsilylhalide. Further, in the process variant (c) for introducing the groups R^{2′} and R^{3′} into the compound of formula (1a), this compound is subjected to an etherification with an alkylhalide in the presence of base, or is treated with thionyl chloride, oxalyl chloride, or phosphorus pentachloride to produce a dichlorophosphonate derivative which is then reacted with a suitable alcohol or amine to give the desired compound.

The phosphonate compound of formula (2) used as a starting material in the above process includes two asymmetric carbons therein, and so has four stereoisomers, each of which is also a novel compound. Therefore, it is another object of the present invention to provide preparation process of the compound of formula (2).

The preparation process of the compound of formula (2) can be characterized in that,

(a) an ethylglycolate, the alcohol group of which is protected, as represented by the following formula (6):

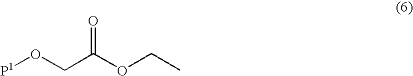

in which P¹ represents an alcohol-protecting group, preferably, benzyl(Bn), tetrahydropiranyl(THP), t-butydiphenylsilyl(TBDPS), or t-butyldimethylsilyl(TBDMS), is reacted with alkyl magnesium halide represented by the following formula (7):
R⁷—MgX (7)

in which R⁷ represents C₃-C₇ alkyl and X represents halogen, in the presence of titanium tetraisopropoxide[Ti(OiPr)₄],

(b) the resulting two cyclopropanol diastereoisomers represented by the following formulae (8) and (9):

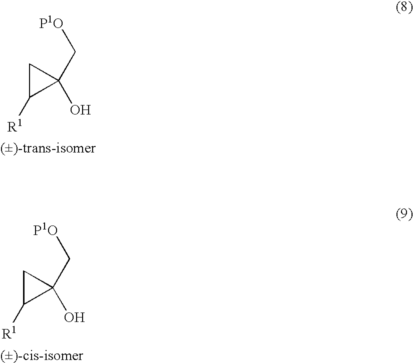

in which R¹ and P¹ are defined as previously described, are separated with a silica gel column,

(c) each compound separated in the step (b) is subjected to an etherification in the presence of base with a compound represented by the following formula (10):

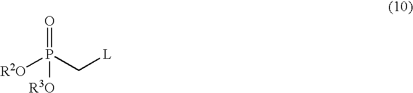

in which R², R³ and L are defined as previously described, to produce a phosphonate compound represented by the following formula (11) or (12), respectively:

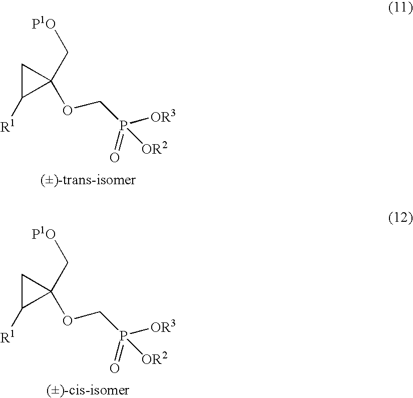

in which R¹, R², R³ and P¹ are defined as previously described, and

(d) the alcohol-protecting group of the resulting compound of formula (11) or (12) is removed and a leaving group(L) is introduced to produce a compound represented by the following formula (2a) or (2b), respectively:

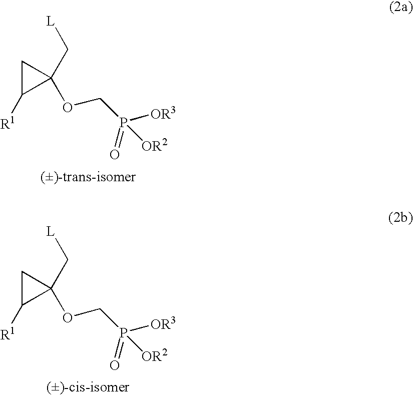

in which L, R¹, R² and R³ are defined as previously described.

Particularly, the compound of formula (2) wherein R¹ is methyl, ethyl, or propyl, butyl and, pentyl and each of R² and R³ is ethyl or isopropyl can be prepared as follows: (i) an ethylglycolate, the alcohol group of which is protected, [compound (6) in Reaction Scheme 1], is reacted with C₃-C₇-alkyl magnesium bromide or C₃-C₇-alkyl magnesium chloride [compound (7) in Reaction Scheme 1] in the presence of titanium tetraisopropoxide[Ti(OiPr)₄], (ii) the resulting two cyclopropanol diastereoisomers [compounds (8) and (9) in Reaction Scheme 1] are separated with a silica gel column, and then each separated compound is subjected to the ether-forming reaction with dialkylhalomethyl phosphonate [compound (10) in Reaction Scheme 1] to produce a phosphonate compound [compounds (11) and (12) in Reaction Scheme 1], (iii) the alcohol-protecting group of the resulting compound is removed and a leaving group (L) is introduced to produce the compound of formulae (2a) and (2b) [compounds (2a) and (2b) in Reaction Scheme 1] (c.f., Reaction Scheme 1):

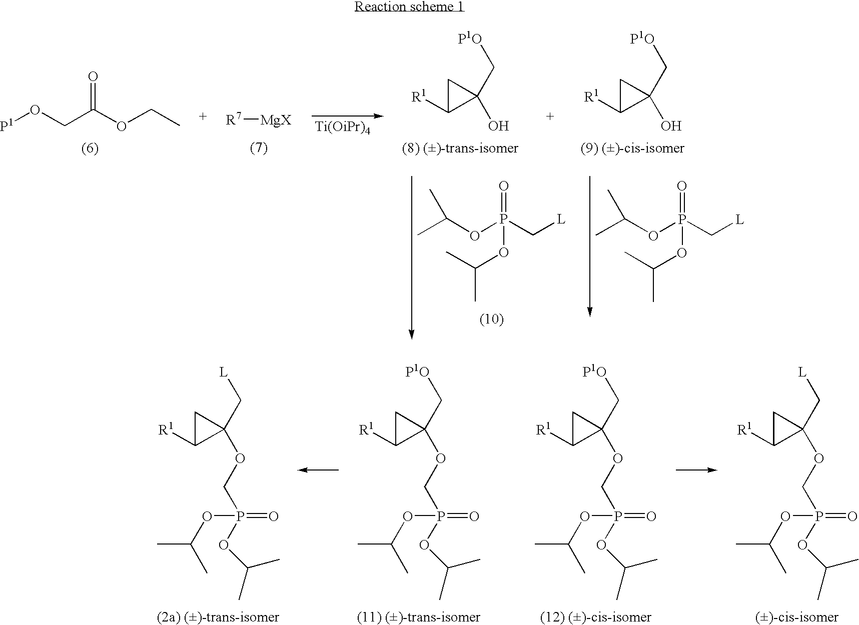

wherein P¹, R⁷, X, R¹ and L are defined as previously described.

Another object of the present invention is to provide processes for the preparation of enantiomers of the compounds of formula (1).

The preparation processes of enantiomer of the compound of formula (1) can be characterized in that,

(a) the compound of formula (1) is resolved to produce its enantiomers by a chiral columm or a chiral reagent; or

(b) a compound represented by the following formula (13) or (14):

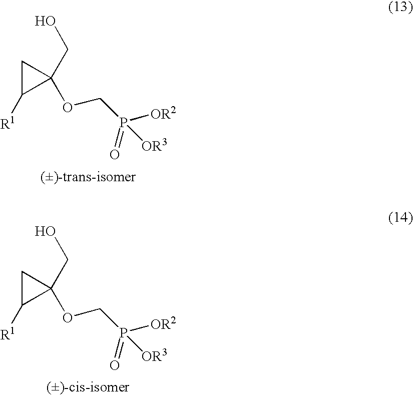

in which R¹, R² and R³ are defined as previously described, is resolved with a hydrolase to produce compounds represented by the following formula (13a) and (13b), or (14a) and (14b), respectively:

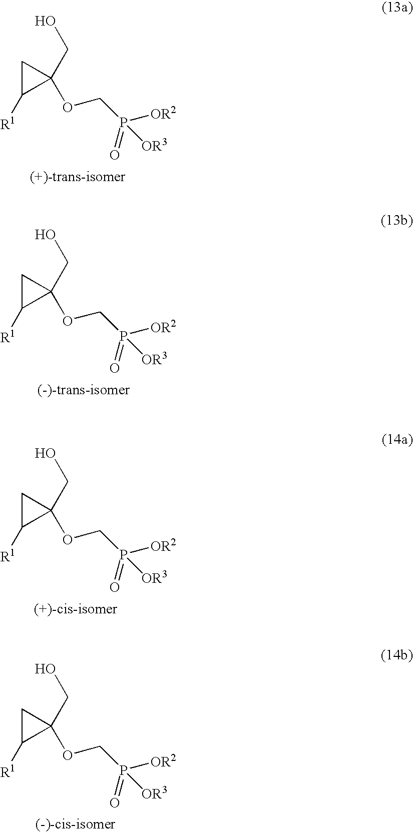

in which R¹, R² and R³ are defined as previously described, and the alcohol group in the resulting each compound is replaced with a leaving group (L), and thereafter the each compound thus obtained is reacted with the compound of formula (3) to produce the enantiomer of the compound of formula (1); or

(c) the compound of formulae (13a), (13b), (14a), or (14b) is prepared through an enantioselective synthesis and is converted to the enantiomer of compound of formula (1) by the procedure described in the process variant (b).

Particularly, the processes variants (a) to (c) might be explained in more detail as follows:

(a) a compound represented by the following formula (4a) or (4b):

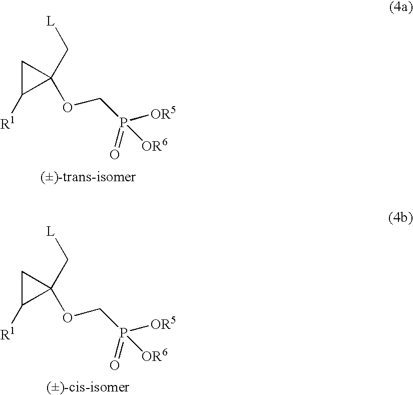

in which R¹, R⁵, R⁶ and L are defined as previously described, is reacted with the compound of formula (3), and the product thus obtained is resolved by a chiral column to produce (+), (−) two optical isomers, each of which is presented as an enantiomer enriched isomer, and the optical isomer is treated with trimethylsilylbromide (TMSBr) to produce the corresponding (+), (−) two optical isomers of the compound of formula (1a), and if necessary, groups R^{2′} and R^{3′} are introduced into the compounds thus obtained to produce the corresponding optical isomers of the compound of formula (1b); or

(b) each of the compound of formula (13) or (14) that is obtained by removing an alcohol-protecting group in the compound of formula (11) or (12) is resolved with a hydrolase (lipase) to produce the corresponding enantiomer enriched compound of formula (13a) and (13b), or (14a) and (14b), and further an alcohol group in the compound of formula (13a), (13b), (14a) or (14b) thus obtained is replaced with a leaving group (L) to produce compound represented by the following formula (2aa), (2ab), (2ba) or (2bb):

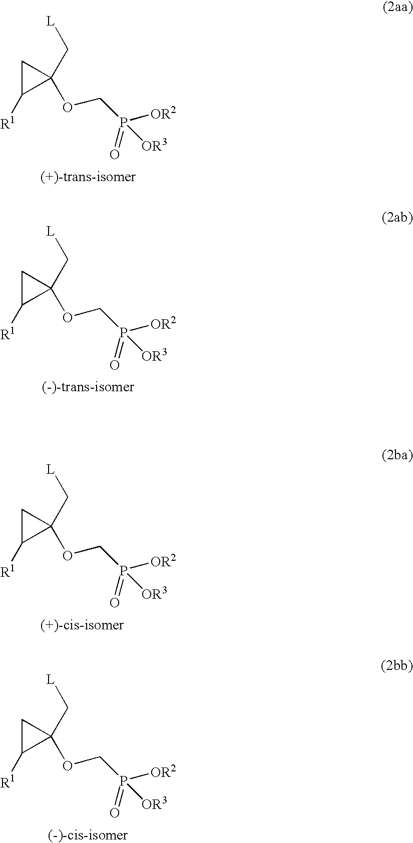

in which R¹, R², R³ and L are defined as previously described, and the resulting compound is reacted with the compound of formula (3) to produce the enantiomer enriched compound of formula (1); or

(c) also the compounds of formulae (13a), (13b), (14a) or (14b) is prepared through an enantioselective synthesis from (+)-(methylenecyclopropyl)carbinol or (−)-(methylenecyclopropyl)carbinol whose absolute configuration is known as follows:

aa) an alcohol-protecting group (P²) is introduced into (+)-(methylenecyclopropyl)carbinol or (−)-(methylenecyclopropyl)carbinol;

bb) the resulting compound is subjected to an dihydroxylation reaction,

cc) an alcohol-protecting group (P¹) is introduced into the primary hydroxy group in the compound obtained in the above (bb) step, and an alcohol-protecting group (P³) is introduced into the tertiary hydroxy group to produce a compound represented by the following formulae (15a), (15b), (16a) or (16b):

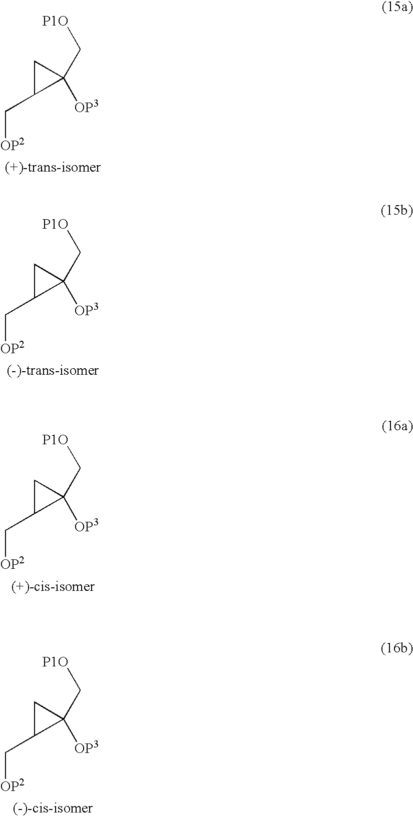

in which P¹ is defined as previously described, P² represents an alcohol-protecting group, preferably benzyl, benzoyl, 4-methoxybenzyl, methyloxybenzoyl, methyloxymethyl or trityl, and P³ represents an alcohol-protecting group, preferably ester group including 1-methoxyacetyl, acetyl, 2-(trimethylsilyl)-1-ethanesulfonyl, etc.,

dd) the protecting group P² in the resulting compound is removed selectively, a leaving group (L) is introduced, and the compound thus obtained is subjected to a reduction with hydrogen or substitution with C₁-C₇-alkyl group,

ee) the protecting group (P³) in the product thus obtained in the above dd) step is removed to produce a compound represented by the following formulae (8a), (8b), (9a) or (9b):

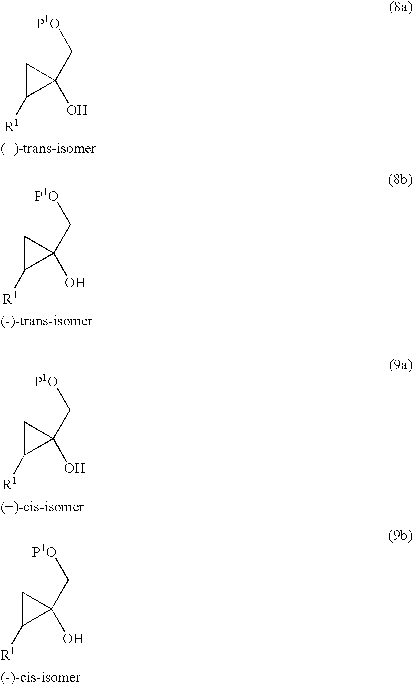

in which R¹ and P¹ are defined as previously described,

ff) the resulting compound in the above step ee) is reacted with the phosphonate compound of formula (10), and the protecting group (P¹) of the compound thus obtained is removed to produce the compound of formulae (13a), (13b), (14a) or (14b),

gg) an alcohol group of each resulting compound is replaced with the leaving group (L) to produce the compound of formulae (2aa), (2ab), (2ba) or (2bb); and

hh) the resulting compound is reacted with the compound of formula (3) to produce the enantiomer enriched compound of formula (1).

The preparation process variants (a) to (c) of the enantiomer of formula (1) can be specifically exemplified by the following Reaction Schemes 2, 3 and 4.

Reaction Scheme 2 is briefly explained below. The compound of formula (2) [compound (4a) in Reaction Scheme 2] is reacted with the compound of formula (3) under the reaction condition as previously described to give the compound of formula (5) [compound (5a) in Reaction Scheme 2]. The resulting compound is resolved by a chiral column to give two enantiomer enriched compound [compounds (5b) and (5c) in Reaction Scheme 2]. The specific rotation of each compound thus obtained is observed to identify (+)-trans-optical isomer(5b) and (−)-trans-optical isomer(5c). Each of optical isomers is treated with trimethylsilylbromide(TMSBr) to give the corresponding enantiomer enriched compounds [compounds (1c) and (1d) in Reaction Scheme 2] of the compound of formula (1a).

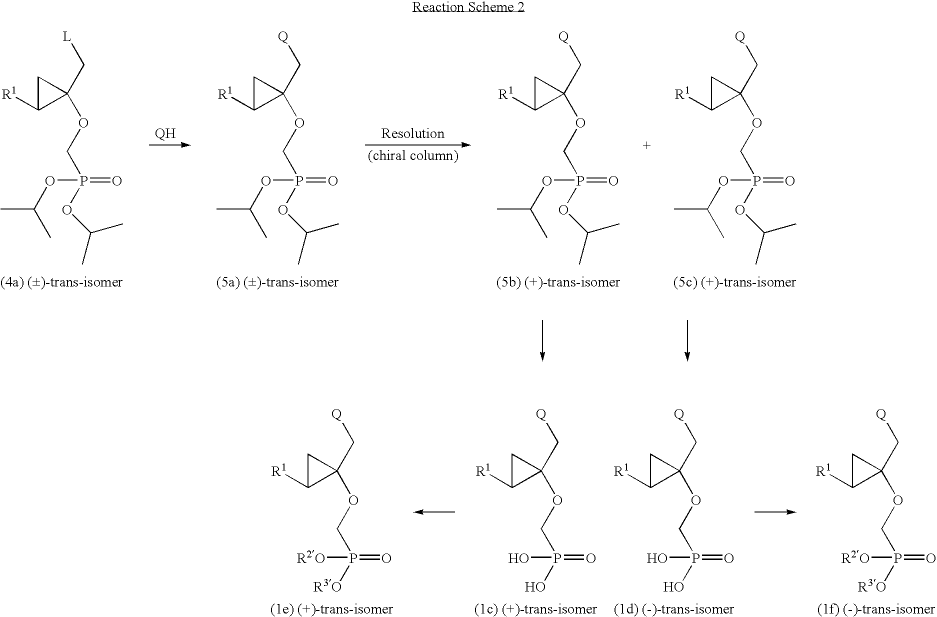

wherein L, R¹, Q, R^{2′} and R^{3′} are defined as previously described.

Reaction Scheme 3 is briefly explained below. The enantiomer enriched compounds [compounds (1c) and (1d) in Reaction Scheme 3] of the compound of formula (1a) can be prepared by using a hydrolase (lipase). An alcohol-protecting group of the compound of formula (11) [compound of formula (11) in Reaction Scheme 3] is removed to give the compound of formula (13) [compound (13) in Reaction Scheme 3]. The compound of formula (13) [compound (13) in Reaction Scheme 3] is subjected to the acylation reaction selectively in non-aqueous organic solvent(s) and in the presence of acylation reagent(s) by using the hydrolase (lipase) to give the compounds of formula (13a) [compound (13a) in Reaction Scheme 3] and acylated compound [compound (17) in Reaction Scheme 3]. Further, the acylated compound [compound (17) in Reaction Scheme 3] is hydrolyzed in aqueous solvent(s) by using the hydrolase (lipase) to give the compound of formula (13b) [compound (13b) in Reaction Scheme 3]. The compounds of formulae (13a) and (13b) thus obtained are subjected to the procedures as previously described to give the enantiomer enriched compounds of formula (1a), respectively. The specific reaction conditions of the above processes can be referred to the following preparations.

The hydrolase (lipase) used in the present invention is meant to an esterlase extracted from Pig liver or Canadida rugosa, or lipase extracted from Canadida antanrctica (fraction A and B), Canadida rugosa, Pseudomonas sp., Porcine pancreas, Humicola sp., Thermomyces sp., or Mucor miehei. The acylation reagent used in the present invention is as follows:

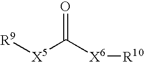

in which R⁹ represents hydrogen, C₁-C₇-alkyl, C₃-C₇-cycloalkyl, or C₅-C₁₀-cycloalkenyl, R¹⁰ represents hydrogen, C₁-C₇-alkyl, or C₁-C₇-alkenyl, and X⁵ and X⁶ independently of one another represent C, O or S.

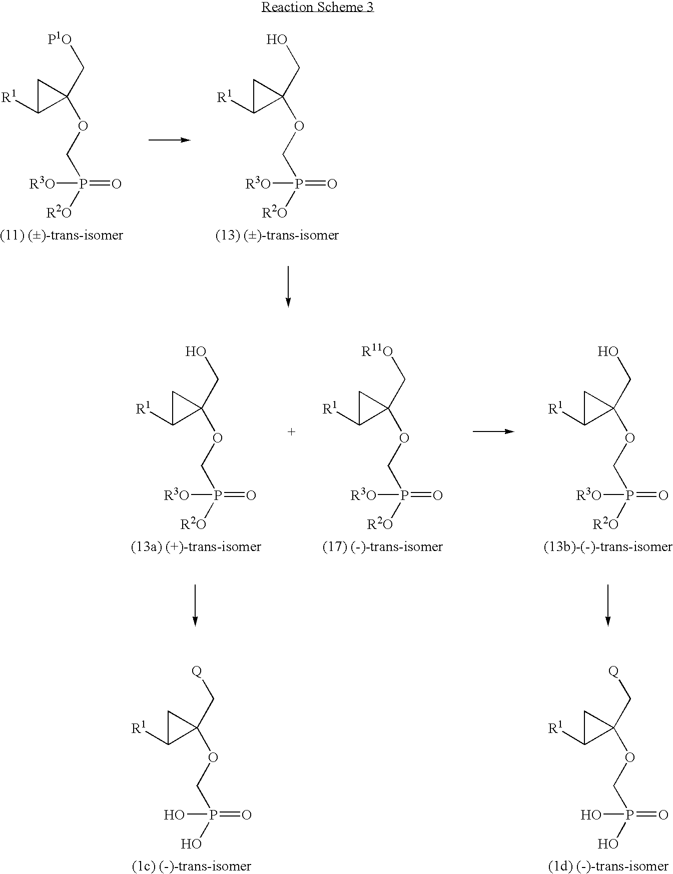

wherein R¹, R², R³, P¹ and Q are defined as previously described, and R¹¹ represents

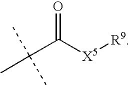

Reaction Scheme 4 is briefly explained below. The enantiomer enriched compound of the compound of formula (1a) [compounds (1c) and (1d) in Reaction Scheme 4] might be prepared through the enantioselective synthesis, another preparation process. By using (+)-(methylenecyclopropyl)carbinol or (−)-(methylenecyclopropyl)carbinol, which is well known chiral compounds, [compound (18) in Reaction Scheme 4] [references: Journal of Organic Chemistry, 67, 286-289 (2002), Journal of Organic Chemistry, 58, 5915-5917 (1993), Journal of Organic Chemistry, 59, 5483-5484 (1994)] as a starting material, the enantiomer enriched compound of the formula (1a) [compound (1c) or (1d) in Reaction Scheme 4] can be prepared as described in Reaction Scheme 4. A protecting group (P²) is introduced into an alcohol group of (+)-(methylenecyclopropyl)carbinol or (−)-(methylenecyclopropyl)carbinol [compound (18) in Reaction Scheme 4]. Two hydroxyl groups are introduced into a double bond in the resulting compound [compound (19) in Reaction Scheme 4], and other protecting groups (P¹ and P³) are selectively introduced into each hydroxyl group to give the compound of formula (15a) or (15b) [compound (20) in Reaction Scheme 4], respectively. The protecting group (P²) of the compound thus obtained [compound (20) in Reaction Scheme 4] is removed selectively to give the alcoholic compound [compound (21) in Reaction Scheme 4] and the hydroxyl group of the resulting compound [compound (21) in Reaction Scheme 4] is replaced with the leaving group (L) to give the compound [compound (22) in Reaction Scheme 4]. The compound thus obtained is subjected to the reductive reaction by using hydrogen, or to the alkyl substitution reaction by using R⁸—M (R⁸ represents C₁-C₆-alkyl and M represents a metal compound including MgBr and Li) to give the compound [compound (23) in Reaction Scheme 4]. The protecting group (P³) of the compound (compound (23) in Reaction Scheme 4) is removed to give the compound of formula (8a) or (8b) [compound (24) in Reaction Scheme 4]. The compound of formula (8a) or (8b) [compound (24) in Reaction Scheme 4] is subjected to the etherification with the compound of formula (10) (dialkyl halomethylphosphonate) and the alcohol-protecting group (P¹) is removed to obtain the enantiomer enriched compound of formula (13a) or (13b) [compound (13a) or (13b) in Reaction Scheme 4]. The compound of formula (13a) or (13b) can be converted to the enantiomer enriched compound of formula (1a) [compound (1c) or (1d) in Reaction Scheme 4] through the same procedure as previously described. The specific reaction conditions of the above process can be referred to the following preparations.

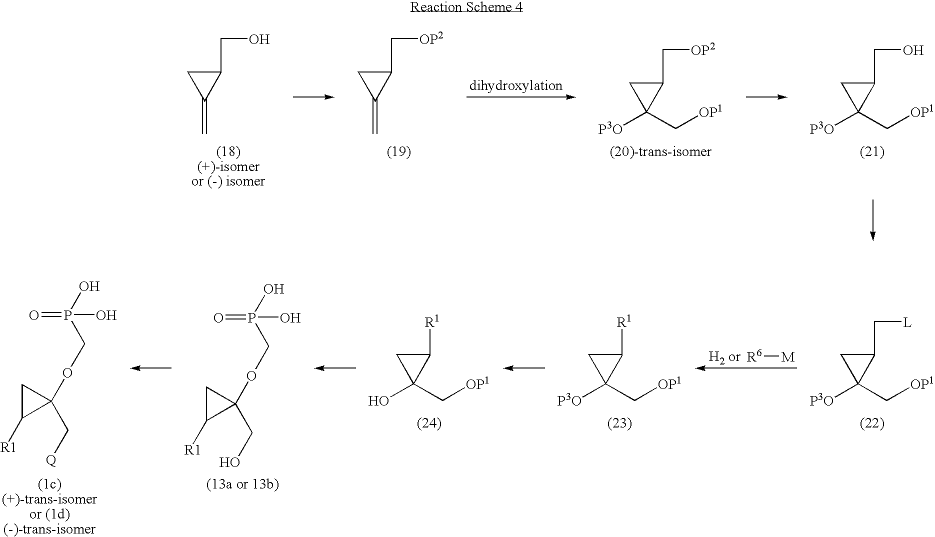

wherein P¹, P², P³, L, R¹ and Q are defined as previously described.

The conditions that are used in the preparation processes and the separation and resolution processes of the compounds according to the present invention, for example, reactants, solvents, bases, amounts of the reactants used, silica gel column, chiral column, eluents, etc., are not restricted to those explained herein. The compounds of the present invention may be also conveniently prepared, and separated and resolved by optionally combining the various synthetic ways, and the separation and resolution methods described in the present specification or known in the arts, and their combinations can be easily performed by one of ordinary skill in the art to which the present invention pertains.

The specific reaction conditions of the above processes can be referred to the following Preparations and Examples.

After the reaction is completed, the resulting product may be further separated and purified by usual work-up processes, such as chromatography, recrystallization, distillation, etc.

(+)-Trans-isomer of the compound of formula (1) of the present invention can be effectively used as antiviral agents. Therefore, another object of the present invention is to provide a composition for the treatment of viral diseases (particularly, against hepatitis B virus), which comprises as an active ingredient (+)-trans-isomer of the compound of formula (1), pharmaceutically acceptable salt, hydrate or solvate thereof together with the pharmaceutically acceptable carrier(s).

When the active compounds according to the present invention are used for clinical purpose, they are preferably administered in an amount ranging generally from 0.01 to 10000 mg, preferably from 0.05 to 100 mg per kg of body weight a day. The total daily dosage may be administered once or over several times. However, the specific administration dosage for a patient can be varied with the specific compound used, the subject patient's body weight, sex, or hygienic condition, diet, the time or method of administration, excretion rate, mixing ratio of agents, severity of a disease to be treated, etc.

The compounds of the present invention may be administered in the form of injections or oral preparations.

Injections, such as sterilized aqueous or oily suspension for injection, can be prepared according to the known procedure using suitable dispersing agent, wetting agent, or suspending agent. The solvents which can be used for preparing injections include water, Ringer's fluid, and isotonic NaCl solution, and also sterilized fixing oil may be conveniently used as the solvent or suspending media. Any non-stimulative fixing oil including mono-, di-glyceride may be used for this purpose, too. Fatty acid such as oleic acid may be also used for injections.

As the solid preparation for oral administration, capsules, tablets, pills, powders, granules, etc., preferably capsules and tablets, can be mentioned. It is also desirable for tablets and pills to be formulated into enteric-coated preparation. The solid preparations may be prepared by mixing the active compound of (+)-trans-isomer of the compound of formula (1) according to the present invention with at least one carrier selected from a group consisting of inactive diluents, such as sucrose, lactose, starch, etc., lubricants such as magnesium stearate, disintegrating agent, and binding agent.

When the compounds according to the present invention are clinically applied for obtaining the desired antiviral effect, the active compound of (+)-trans-isomer of the compound of formula (1) can be administered in combination with one or more substances selected from the known anti-cancer or antiviral agents. As the anti-cancer or antiviral agents which can be administered together with the compound of the present invention in such a manner, 5-Fluorouracil, Cisplatin, Doxorubicin, Taxol, Gemcitabine, Lamivudine, etc. can be mentioned.

However, the preparations comprising the compound of the present invention are not restricted to those explained above, and may contain any substance useful for the treatment or prevention of cancers or viral diseases.

The present invention will be more specifically explained in the following Preparations, Examples, and Experiments. However, it should be understood that these Preparations, Examples, and Experiments are intended to illustrate the present invention but not in any manner to limit the scope of the present invention.

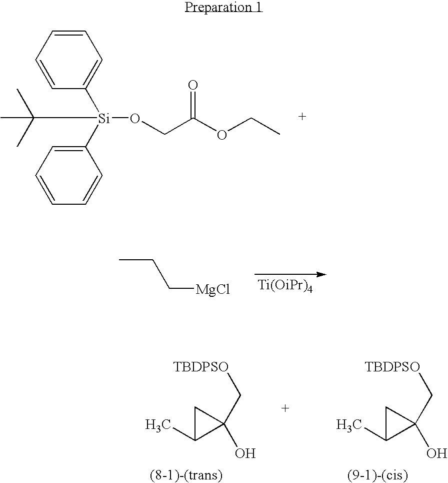

### Synthesis of (±)-trans-1-({[t-butyl(diphenyl)silyl]oxy}methyl)-2-methylcyclopropanol(8-1) and (±)-cis-1-({[t-butyl(diphenyl)silyl]oxy}methyl)-2-methylcyclopropanol (9-1)

According to the description in a reference (see: Syn. Lett. 07, 1053-1054, 1999), the title compound was prepared as follows: 50 g (0.146 mole) of ethyl 2-{[t-butyl(diphenyl)silyl]oxy}acetate was dissolved in 700 ml of tetrahydrofuran (THF), and 30 ml of titaniumtetraisopropoxide was added thereto. To the mixture was slowly added 290 ml of propylmagnesiumchloride (2.OM in THF) at −15° C., and the reaction solution was stirred for 12 hours at ambient temperature. 50 ml of saturated ammonium chloride was added to stop the reaction. About 700 ml of tetrahydrofuran(THF) used as a solvent was removed by distillation under reduced pressure, and the reaction mixture was extracted twice with 700 ml of hexane. The hexane extract was distilled under reduced pressure, and the residue was separated by a silica gel column (eluent: 1:8/ethylacetate: hexane) to give two title compounds (diastereoisomers: diastereoisomers), 38 g (8-1) and 3.8 g (9-1). The structure of each compound was confirmed by NMR.

Title compound(8-1)

¹H NMR(CDCl₃) δ0.08 (t, 1H), 0.90 (q, 1H), 0.96 (d, 3H), 1.08 (s, 9H), 1.14 (m, 1H), 2.79 (s, 1H), 3.70 (d, 1H), 3.84 (d, 1H), 7.43 (m, 6H), 7.70(m, 4H)

ESI: 363 (M+Na)⁺, C21H28O2Si

Title compound(9-1)

¹H NMR(CDCl₃) δ0.31 (t, 1H), 0.62 (q, 1H), 0.69 (m, 1H), 1.07 (s, 9H), 1.15 (d, 3H), 2.46 (s, 1H), 3.49 (d, 1H), 3.79 (d, 1H), 7.43 (m, 6H), 7.70(m, 4H)

ESI: 363 (M+Na)⁺, C21H28O2Si

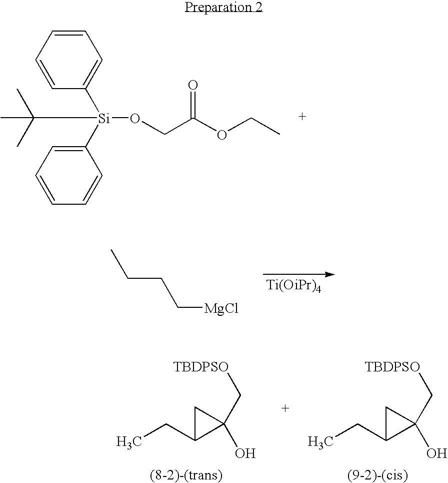

### Synthesis of (±)-trans-1-({[t-butyl(diphenyl)silyl]oxy}methyl)-2-ethylcyclopropanol(8-2) and (±)-cis-1-({[t-butyl(diphenyl)silyl]oxy}methyl)-2-ethylcyclopropanol (9-2)

The same procedure as Preparation 1 was conducted, but butylmagnesiumchloride was used instead of propylmagnesiumchloride to give 30 g of the title compound (8-2) as the main compound, but compound (9-2) was hardly obtained.

Title Compound (8-2)

¹H NMR(CDCl₃) δ0.09 (t, 1H), 0.97 (q, 1H), 0.97 (t, 3H), 1.06 (2H), 1.07 (s,9H) 1.31 (t, 1H), 2.79 (s, 1H), 3.71 (d, 1H), 3.81 (d, 1H), 7.41 (m, 6H), 7.68(m, 4H)

ESI: 377 (M+Na)⁺, C22H30O2Si

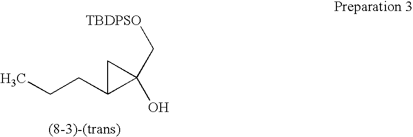

### Synthesis of (±)-trans-1-({[t-butyl(diphenyl)silyl]oxy}methyl)-2-propylcyclopropanol(8-3)

The same procedure as Preparation 1 was conducted, but pentylmagnesiumchloride was used instead of propylmagnesiumchloride to give 25 g of the title compound (8-3) as the main compound.

Title Compound (8-3)

¹H NMR(CDCl₃) 80.09 (t, 1H), 0.68 (1H), 0.70 (t, 3H), 0.82 (m,1H), 1.09 (s, 10H), 1.32 (m, 1H), 1.40 (m, 2H), 2.90 (s, 1H), 3.73 (d, 1H), 3.85 (d, 1H), 7.45 (m, 4H)

ESI: 391 (M+Na)⁺, C23H32O2Si

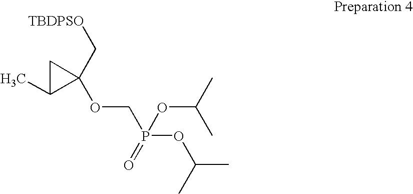

### Synthesis of diisopropyl {(±)-trans-1-({[t-butyl(diphenyl)silyl]oxy}methyl)-2-methylcyclopropyl}oxy}methylphosphonate

The compound (8-1) prepared in Preparation 1 (7.5 g) was dissolved in 35 ml of dimethylformamide and 9.7 g of diisopropyl bromomethylphosphonate was added thereto, and the resulting mixture was stirred for 10 minutes. To the mixture was slowly added 35 ml of lithium t-butoxide(1.0M in THF) at 50° C., and the mixture was stirred for 4 hours more. Dimethylformamide was removed by distillation under reduced pressure, 40 ml of saturated ammonium chloride was added to the residue, which was then extracted with ethyl acetate. The ethyl acetate extract was distilled under reduced pressure, and the residue was purifies by a silica gel column chromatography (eluent: ethylacetate/n-hexane=1/1, v/v ) to give 7.0 g (yield 61%) of the title compound.

¹H NMR(CDCl₃) δ0.13 (t, 1H), 0.96 (m, 1H), 0.97 (d, 3H), 1.05 (m, 1H), 1.06 (s, 9H), 1.30 (t, 12H), 3.70 (d, 1H), 3.98 (d, 2H), 4.00 (d, 1H), 4.75 (m, 2H), 7.42 (m, 6H), 7.70 (m, 4H)

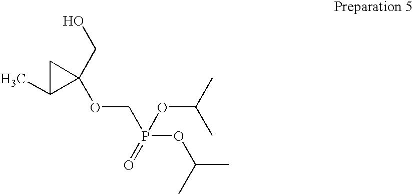

### Synthesis of diisopropyl {(+)-trans-1-(hydroxymethyl)-2-methylcyclopropyl}oxy}methylphosphonate

The compound prepared in Preparation 4 (8.3 g) was dissolved in 100 ml of methanol, 3.1 g of ammonium fluoride was added thereto, and the resulting mixture was heated under reflux for 2 hours. After the reaction was completed, methanol was removed by distillation under reduced pressure, and the residue was purified by a silica gel column chromatography (eluent: dichloromethane/methanol=20/1, v/v) to give 3.6 g (yield 82%) of the title compound.

¹H NMR(CDCl₃) δ0.23 (t, 1H), 0.96 (dd, 1H), 1.12 (d, 3H), 1.23 (m, 1H), 1.32 (d, 12H), 3.59 (d, 1H), 3.82 (d, 2H), 3.96 (d, 1H), 4.01 (s, 1H), 4.82 (m, 2H)

ESI: 303 (M+Na)⁺, C12H25O5P

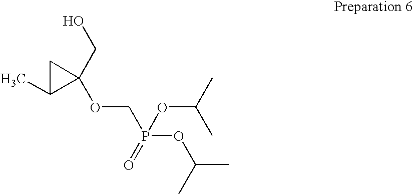

### Synthesis of diisopropyl {(±)-cis-1-(hydroxymethyl)-2-methylcyclopropyl]oxy}methylphosphonate

The compound (9-1) prepared in Perparation 1 (3.0 g) was consecutively reacted according to the same procedure as Preparations 4 and 5 to give 1.2 g of the title compound.

¹H NMR(CDCl₃) δ0.41 (t, 1H), 0.71 (dd, 1H), 0.89 (m, 1H), 1.13 (d, 3H), 1.33(d, 12H), 3.50 (m, 1H), 3.65 (m, 1H), 3.81 (dd, 1H), 3.91 (dd, 1H), 4.29 (s, 1H), 4.76 (m, 2H)

ESI: 303 (M+Na)⁺, C12H25O5P

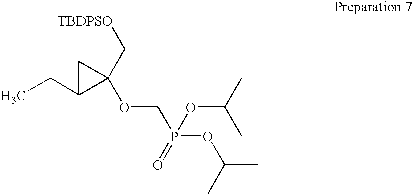

### Synthesis of diisopropyl {(±)-trans-1-({[t-butyl(diphenyl)silyl]oxy}methyl)-2-ethylcyclopropyl}oxy}methylphosphonate

The compound (8-2) prepared in Preparation 2 (4.2 g) was reacted according to the same procedure as Preparation 4 to give 3.6 g of the title compound.

¹H NMR(CDCl₃) δ0.15 (t, 1H), 0.92 (m, 1H), 0.94 (t, 3H), 1.06 (s, 9H), 1.08 (m, 1H), 1.25 (m, 1H), 1.31(m, 12H), 1.35 (m,1H), 3.73 (d, 1H), 3.98 (m, 3H), 4.74 (m, 2H), 7.41 (m, 6H), 7.67 (m, 4H)

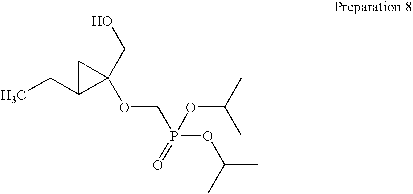

### Synthesis of diisopropyl {(±)-trans-1-(hydroxymethyl)-2-ethylcyclopropyl]oxy}methylphosphonate

The compound prepared in Preparation 7 (3.6 g) was reacted according to the same procedure as Preparation 5 to give 1.6 g of the title compound.

¹H NMR(CDCl₃) δ0.27 (t, 1H), 0.95 (dd, 1H), 1.02 (d, 3H), 1.15 (m, 1H), 1.29 (m, 1H), 1.34 (d, 12H), 1.37 (m, 1H), 3.68 (dd, 1H), 3.84 (d, 2H), 3.88 (dd, 1H), 4.00 (brt, 1H), 4.77 (m, 2H).

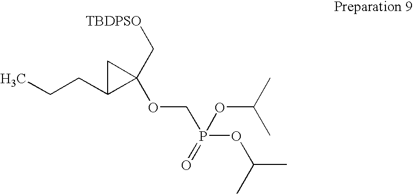

### Synthesis of diisopropyl {(±)-trans-1-({[t-butyl(diphenyl)silyl]oxy}methyl)-2-propylcyclopropyl}oxy}methylphosphonate

The compound (8-3) prepared in Preparation 3 (1.2 g) according to the same procedure as Preparation 4 to give 1.1 g of the title compound.

¹H NMR(CDCl₃) δ0.14 (t, 1H), 0.85 (t, 3H), 0.95 (m, 2H), 1.05 (s, 9H), 1.25 (m, 1H), 1.31(m, 12H), 1.38 (m,3H), 3.70 (d, 1H), 3.98 (m, 3H), 4.72 (m, 2H), 7.38 (m, 6H), 7.66 (m, 4H).

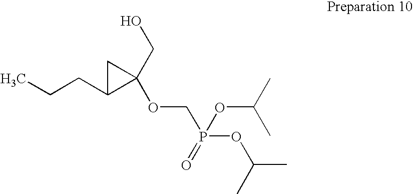

### Synthesis of diisopropyl {(±)-trans-1-(hydroxymethyl)-2-propylcyclopropyl}oxy}methylphosphonate

The compound prepared in Preparation 9 (1.2 g) according to the same procedure as Preparation 5 to give 0.5 g of the title compound.

¹H NMR(CDCl₃) δ0.28 (t, 1H), 0.94 (t, 3H), 0.97 (m, 1H), 1.20 (m, 2H), 1.33 (d, 12H), 1.41 (m, 3H), 3.65 (dd, 1H), 3.82 (d, 2H), 3.87 (dd, 1H), 4.00 (brt, 1H), 4.77 (m, 2H),

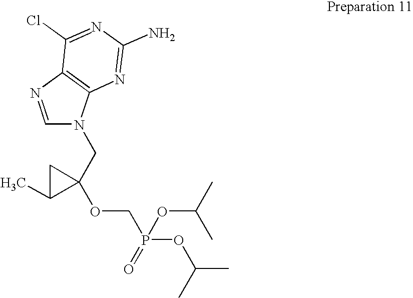

### Synthesis of diisopropyl ({(±)-trans-1-[(2-amino-6-chloro-9H-purine-9-yl)methyl]-2-methylcyclopropyl}oxy}methylphosphonate

The compound prepared in Preparation 5 (2.3 g) was dissolved in 75 ml of dichloromethane, 1.23 g of triethylamine and 1.2 g of methanesulfonylchloride were added thereto, and the resulting mixture was stirred for 30 minutes at room temperature. Saturated ammonium chloride was added to stop the reaction. The product was extracted with dichloromethane, and the dichloromethane was removed by distillation under reduced pressure to give 2.73 g (yield 94%) of methanesulfonate compound, which was used in the next reaction without any purification.

¹H NMR(CDCl₃) δ0.44 (t, 1H), 1.16 (d, 3H), 1.20 (m, 1H), 1.32 (m, 12H), 1.30 (m, 1H), 3.14 (s, 3H), 3.82 (m, 2H), 4.33 (d, 1H), 4.68 (d, 1H), 4.78 (m, 2H).

The methanesulfonate thus obtained (430 mg) was dissolved in 18 ml of dimethylformamide, and 57.6mg (60% purity) of sodium hydride and 162 mg of 6-chloroguanine (2-amino-6-chloro-9H-purine) were added thereto. The reaction mixture was refluxed under heating for 4 hours. Saturated ammonium chloride was added to stop the reaction. The product was extracted with ethyl acetate, and the ethyl acetate extract was distilled under reduced pressure, and the residue was purified by a silica gel column chromatography (eluent: dichloromethane/methanol=20/1, v/v) to give 201 mg (yield 44%) of the title compound.

¹H NMR(CDCl₃) δ0.50 (t, 1H), 1.12 (m, 1H), 1.16 (d, 3H), 1.21(dd 6H), 1.27 (t, 6H), 1.39 (m, 1H), 3.86 (m, 2H), 4.31 (d, 2H), 4.69 (m, 2H), 5.13 (brs, 2H), 8.32 (s, 1H)

ESI: 432 (M+1)⁺, C17H27C1N5O4P

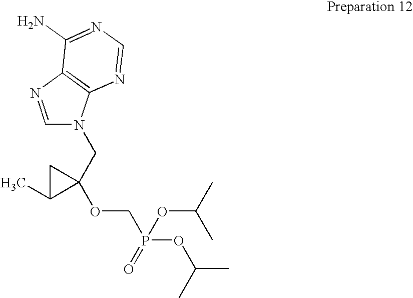

### Synthesis of diisopropyl ({(±)-cis-1-[(6-amino-9H-purine-9-yl)methyl]-2-methylcyclopropyl}oxy)methylphosphonate

The compound prepared in Preparation 6 (0.51 g) was reacted according to the same procedure as Preparation 11, except that adenine was reacted instead of 6-chloroguanine, to give 250 mg of the title compound.

ESI: 398(M+1)⁺, C17H28N5O4P

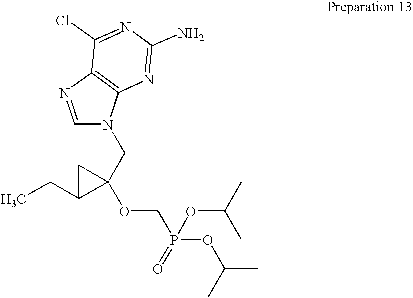

### Synthesis of diisopropyl ({(±)-trans-1-[(2-amino-6-chloro-9H-purine-9-yl)methyl]-2-ethylcyclopropyl}oxy)methylphosphonate

The same procedure as Preparation 11 was conducted to the compound prepared in Preparation 8 (620 mg) to give 330 mg of the title compound.

¹H NMR(CDCl₃) δ0.53 (t, 1H), 0.97 (t, 3H), 1.08(m, 1H), 1.25(dd 6H), 1.26 (m, 1H), 1.28 (t, 6H), 1.40 (m, 2H), 3.80 (m, 2H), 4.16 (d, 1H), 4.40 (d, 1H), 4.69 (m, 2H), 5.10 (s, 2H), 8.18 (s, 1H).

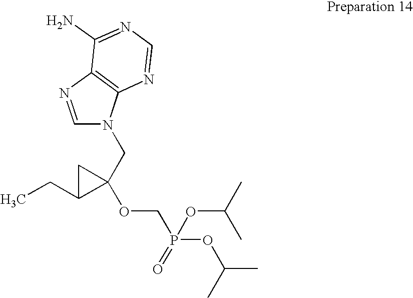

### Synthesis of diisopropyl ({[(±)-(trans)]-1-[(6-amino-9H-purine-9-yl)methyl]-2-ethylcyclopropyl}oxy)methylphosphonate

The compound prepared in Preparation 8 (210 mg) was reacted according to the same procedure as Preparation 11, except that adenine was reacted instead of 6-chloroguanine, to give 95 mg of the title compound.

¹H NMR(CDCl₃) δ0.58 (t, 1H), 0.98 (t, 3H), 1.12(m, 1H), 1.28(dd 6H), 1.26 (m, 1H), 1.39 (m, 6H), 1.42 (m, 2H), 3.80 (m, 2H), 4.32 (d, 1H), 4.68 (d, 1H), 4.75 (m, 2H), 5.92 (brs, 2H), 8.29 (s, 1H), 8.34 (s, 1H).

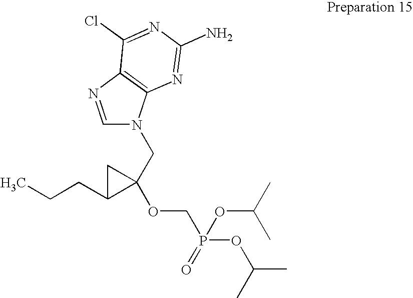

### Synthesis of diisopropyl ({[(±)-(trans)]-1-[(2-amino-6-chloro-9H-purine-9-yl)methyl]-2-propylcyclopropyl}oxy)methylphosphonate

The same procedure as Preparation 11 was conducted to the compound prepared in Preparation 10 (240 mg) to give 110 mg of the title compound.

¹H NMR(CDCl₃) δ0.55 (t, 1H), 0.93 (t, 3H), 1.13(m, 1H), 1.25 (dd 6H), 1.26 (m, 1H), 1.29 (t, 6H), 1.31 (m, 4H), 1.40 (m, 1H), 3.80 (m, 2H), 4.18(d, 1H), 4.40 (d, 1H), 4.69 (m, 2H), 5.06 (s, 2H), 8.18 (s, 1H).

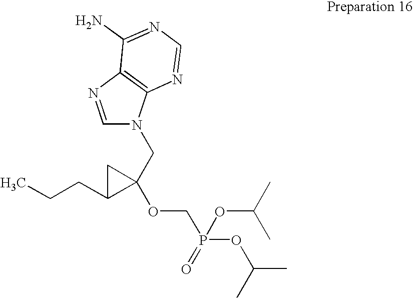

### Synthesis of diisopropyl ({[(±)-(trans)-1-[(6-amino-9H-purine-9-yl)methyl]-2-propylcyclopropyl}oxy)methylphosphonate

The compound prepared in Preparation 10 (105 mg) was reacted according to the same procedure as Preparation 11, except that adenine was reacted instead of 6-chloroguanine, to give 45 mg of the title compound.

¹H NMR(CDCl₃) δ0.59 (t, 1H), 0.91 (t, 3H), 1.12(m, 1H), 1.31(m 12H), 1.32 (m, 5H), 3.80 (m, 2H), 4.32 (d, 1H), 4.50 (d, 1H), 4.72 (m, 2H), 5.80 (brs, 2H), 8.28 (s, 1H), 8.34 (s, 1H).

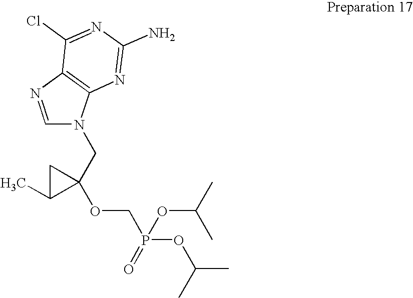

### Synthesis of diisopropyl ({[(±)-(cis)]-1-[(2-amino-6-chloro-9H-purine-9-yl)methyl]-2-methylcyclopropyl}oxy)methylphosphonate

The compound prepared in Preparation 6 (80 mg) was reacted according to the same procedure as Preparation 11 to give 35 mg of the title compound.

ESI: 432 (M+1)⁺, C17H27C1N5O4P

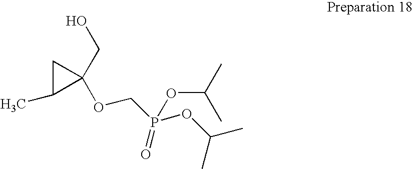

### Synthesis of diisopropyl {[(±)-trans-1-(hydroxymethyl)-2-methylcyclopropyl]oxy}methylphosphonate and diisopropyl {[(−)-trans-1-(hydroxymethyl)-2-methylcyclopropyl]oxy}methylphosphonate

The racemate prepared in Preparation 5 (51 g) was dissolved in 200 ml of toluene, 1.5 g of lipase (Canadida antanrctica B, immobilised, Novozyme 435), and 11.8 ml of vinyl acetate were added thereto, and the resulting mixture was stirred for 40 hours at ambient temperature. The solvent was removed by distillation under reduced pressure, and the mixture compounds (13a) and (17) were separated by a chromatography method to give 17.7 g of the compound (13a) and 38.4 g of the compound (17). The compound (17) was added to 100 ml of phosphate buffer (0.3M, pH 7.2), the solution was hydrolyzed by 1.54 g of Novozyme 435 as much as 60%, and extracted with an organic solvent. And, the solvent was removed by distillation under reduced pressure, and the mixture was separated to give 16.6 g of the compound [the compound (13b) in Reaction Scheme 3] and 18.92 g of the compound [the compound (17) in Reaction Scheme 3]. The compound [the compound (17) in Reaction Scheme 3] was hydrolyzed according to the same procedure as the above, and the resulting mixture was separated to give 6.2 g of the compound [the compound (13b) in Reaction Scheme 3] and 8.3 g of the compound [the compound (17) in Reaction Scheme 3]. 8.3 g of the compound [the compound (17) in Reaction Scheme 3] thus obtained was completely hydrolyzed by the same procedure as the above to give 8 g of the compound (13a). The optical activities (specific rotation) of the two compounds, compounds (13a) and (13b) in Reaction Scheme 3, were [α]_{D}=+42.27 and −46.50, respectively. To determine the optical purity of the above two compounds, the purity of the products prepared by reacting the above two compound [compounds (13a) and (13b) in Reaction Scheme 3] with s-(+)methoxyphenylacetylchloride in the presence of a base was confirmed by the high pressure liquid chromatography (HPLC, using chiral column). The resulting optical purity of the two compounds [compound (13a) and compound (13b) in Reaction Scheme 3] was over 95% for both. The retention time of the compound induced from the compound (13a) in Reaction Scheme 3 was 13 minutes and that of the compound induced from the compound (13b) in Reaction Scheme 3 was 14 minutes (0.9 ml/min, Hexane: isopropanol, 95:5).

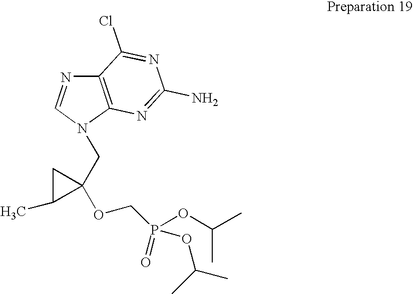

### Synthesis of diisopropyl({(+)-(trans)-1-[(2-amino-6-chloro-9H-purine-9-yl)methyl]-1-methylcyclopropyl}oxy)methylphpsphonate and diisopropyl ({(−)-(trans)-1-[(2-amino-6-chloro-9H-purine-9-yl)methyl]-1-methylcyclopropyl}oxy)methylphpsphonate.

The compound [the compound (13a) in Reaction Scheme 3] prepared in Preparation 18 was reacted according to the same procedure as Preparation 11 to give the desired title compound. ¹H NMR, Mass and optical activity were the same as those of the compound (5b-1) prepared in Example 1. Furthermore, ¹H NMR , Mass and optical activity of the compound obtained by applying the same method to the compound (13b) prepared in Preparation 18 were the same as those of the compound (5c-1) prepared in Example 1.

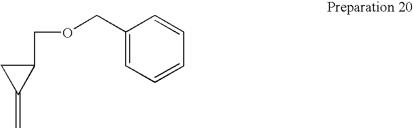

### Synthesis of 1-({[(1R)-2-methylenecyclopropyl]methoxy}methyl)benzene

The well known compound [(2R)-methylenecyclopropyl]methanol (300 mg) [Reference: Journal of Organic Chemistry, 67, 286-289 (2002), Journal of Organic Chemistry, 58, 5915-5917 (1993), Journal of Organic Chemistry, 59, 5483-5484 (1994)] was dissolved in 10 ml of dimethylamide(DMF), 214 mg of sodium hydride (NaH, 60 % in mineral oil) and 732.5 mg of benzyl bromide(BnBr) were added to the solution, and the mixture was stirred for 10 hours. Water (20 ml) and diethylether (100 ml) were added thereto. The organic layer was separated and removed by distillation under reduced pressure, and the residue was purified by a silica gel column chromatography (eluent: ethylacetate/n-hexane: 5/95, v/v) to give 350 mg (yield 57%) of the title compound.

¹H NMR (CDCl₃) δ0.97 (m, 1H), 1.35 (tt, 1H), 1.80 (m, 1H), 3.17 (dd, 1H), 3.53 (dd, 1H), 4.56 (q, 2 H), 5.47 (br s, 1H), 5.46 (br s, 1H), 7.31 (m, 5H).

ESI: 175 (M+1)⁺, C12H14O.

Furthermore, the same procedure as the above was conducted by using [(2S)-methylenecyclopropyl]methanol as a starting material to give 1-({[(1S)-2-methylene cyclopropyl]methoxy}methyl)benzene, and its NMR data was the same as the title compound.

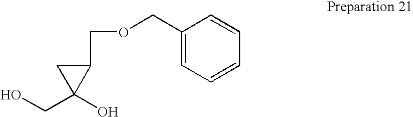

### Synthesis of (1R,2S)-2-[(benzyloxy)methyl]-1-(hydroxymethyl)cyclopropanol

The compound prepared in Preparation 20 (200 mg) was dissolved in water/THF (5 ml/5 ml), and 1 ml of OsO4 (Osmium tetroside, 2.5 wt % solution in t-butanol) and NMO (4-methyl morpholine N-oxide) were added thereto. After stirring the mixture for 24 hours, water (20 ml) and methylenedichloride (50 ml) were added thereto, and the organic layer was separated. The organic layer was removed by distillation under reduced pressure, and the residue was purified by a silica gel column chromatography (eluent: methylenedichloride/methanol: 95/5, v/v) to give 220 mg (yield 92%) of the title compound.

¹H NMR (CDCl₃) δ0.47 (t, 1H), 1.10 (dd, 1H), 1.49 (m, 1H), 2.97 (t, 1H), 3.16 (br d, 1H), 3.40 (d, 1H), 3.67 (br s, 1H), 3.86 (q, 1H), 3.98 (t, 1H), 4.46 (d, 1H), 4.58 (d, 1H), 7.34 (m, 5H).

¹³C NMR (CDCl₃) δ14.9, 22.0, 53.4, 69.0, 69.3, 73.1, 127.8, 127.9 (2C), 128.4 (2C), 137.9.

[α]_{D}=(+)7.7 (c=0.013 in CHCl₃)

ESI: 209 (M+1)⁺, C12H16O3.

Furthermore, the same procedure as the above was conducted by using 1-({[(1S)-2-methylenecyclopropyl]methoxy}methyl)benzene as a starting material to obtain (1S,2R)-2-[(benzyloxy)methyl]-1-(hydroxymethyl)cyclopropanol, and its NMR data was the same as the title compound. The optical activity was [α]_{D}=(−)8.0 (c=0.01 in CHCl₃).

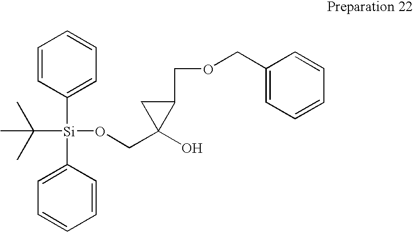

### Synthesis of (1R,2S)-2-[(benzyloxy)methyl]-1-({[tert-butyl(diphenyl)silyl]oxy}methyl)-cyclopropanol

The compound prepared in Preparation 21 (250 mg) was dissolved in DMF (10 ml) and 350 mg of imidazole and 360 mg of diphenyl tert-butylsilylchloride dissolved in DMF (5 ml) were slowly added dropwise thereto at 0□. The resulting mixture was stirred for 10 hours at ambient temperature. Water (20 ml) and diethylether (50 ml) were added thereto. The organic layer therein was separated and removed by distillation under reduced pressure, and the residue was purified by a silica gel column chromatography (eluent: ethylacetate/n-hexane: 1/5, v/v) to give 280 mg (yield 52%) of the title compound.

¹H NMR (CDCl₃) δ0.39 (t, 1H), 1.03 (dd, 1H), 1.08 (s, 9H), 1.52 (m, 1H), 2.83 (s, 1H), 3.27 (dd, 1H), 3.39 (dd, 1H), 3.80 (q, 2H), 4.50 (s, 2H), 7.31 (m, 5H), 7.36 (m, 10H), 7.68 (m, 4H).

¹³C NMR (CDCl₃) δ16.4, 19.3, 24.2, 26.9 (3C), 59.1, 66.6, 69.5, 72.5, 127.5 (2C), 127.6 (2C), 127.8 (4C), 128.3 (2C), 129.8 (2C), 133.2, 133.3, 135.6 (4C), 138.2.

ESI: 447 (M+1)⁺, C28H34O3Si.

Furthermore, the same procedure as the above was conducted by using (1S,2R)-2-[(benzyloxy)methyl]-1-(hydroxymethyl)cyclopropanol as a starting material to obtain (1S,2R)-2-[(benzyloxy)methyl]-1-({[tert-butyl(diphenyl)silyl]oxy}methyl)-cyclopropanol, and its NMR data was the same as the title compound.

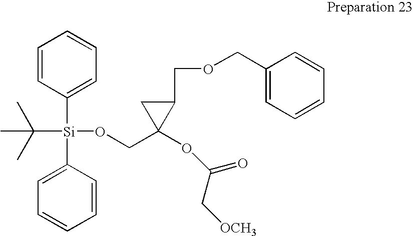

### Synthesis of (1R,2S)-2-[(benzyloxy)methyl]-1-({l [tert-butyl(diphenyl)silyl]oxy}methyl)-cyclopropyl 2-methoxyacetate

The compound prepared in Preparation 22 (250 mg) was dissolved in dichloromethane (10 ml), and 1.0 ml of TEA (triethylamine) and 400 mg of 2-methoxyacetylchloride were slowly added dropwise thereto at 0° C. The resulting mixture was stirred for 10 hours at ambient temperature. Water (20 ml) and diethylether (50 ml) were added thereto. The organic layer therein was separated and removed by distillation under reduced pressure, and the residue was purified by a silica gel column chromatography (eluent: ethylacetate/n-hexane: 1/5, v/v) to give 200 mg (yield 69%) of the title compound.

¹H NMR (CDCl₃) δ0.86 (t, 1H), 1.03 (s, 9H), 1.15 (tt, 1H), 1.57 (m, 1H), 3.34 (dd, 1H), 3.38 (s, 3H), 3.73 (dd, 1H), 3.85 (d, 2H), 3.88 (d, 2H), 4.11 (d, 2H), 4.48 (s, 2H), 7.37 (m, 11H), 7.61 (m, 4H).

¹³C NMR (CDCl₃) δ15.7, 19.2, 23.2, 26.8 (3C), 59.3, 63.2, 64.2, 68.6, 69.6, 72.6, 127.6 (2C), 127.7 (2C), 127.8 (4C), 128.3 (2C), 129.7 (2C), 133.3, 133.4, 135.6 (4C), 138.2, 169.8.

ESI: 519 (M+1)⁺, C31H38O5Si.

Furthermore, the same procedure as the above was conducted by using (1S,2R)-2-[(benzyloxy)methyl]-1-({[tert-butyl(diphenyl)silyl]oxy}methyl)-cyclopropanol as a starting material to obtain (1S,2R)-2-[(benzyloxy)methyl]-1-({[tert-butyl(diphenyl)silyl]oxy}methyl)-cyclopropyl 2-methoxyacetate, and its NMR data was the same as the title compound.

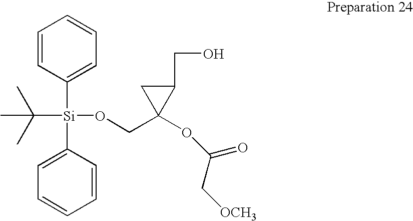

### Synthesis of (1R,2S)-1-({[tert-butyl(diphenyl)silyl]oxy}methyl)-2-(hydroxymethyl)cyclopropyl 2-methoxyacetate

The compound prepared in Preparation 23 (200 mg) was dissolved in methanol (20 ml), and 40 mg of 10% Pd on Carbon was added thereto. The resulting mixture was reduced with hydrogen gas under 1 atm for 24 hours. The 10% Pd on Carbon (50 mg) was further added thereto, and the resulting mixture was additionally reduced for 24 hours. The Pd on Carbon was removed by celite, the residual solution was removed by distillation under reduced pressure, and the residue was purified by a silica gel column chromatography (eluent: ethylacetate/n-hexane: 1/2, v/v) to give 160 mg (yield 98%) of the title compound.

¹H NMR (CDCl₃) δ0.81 (t, 1H), 1.10 (s, 9H), 1.11 (m, 1H), 1.73 (m, 1H), 3.19 (d, 1H), 3.26 (t, 1H), 3.36 (s, 3H), 3.72 (dd, 1H), 3.82 (q, 2H), 3.96 (m, 1H), 4.38 (d, 1H), 7.45 (m, 6H), 7.63 (m, 4H).

ESI: 429 (M+1)⁺, C24H32O5Si.

Furthermore, the same procedure as the above was conducted by using (1S,2R)-2-[(benzyloxy)methyl]-1-({[tert-butyl(diphenyl)silyl]oxy}methyl)-cyclopropyl 2-methoxyacetate as a starting material to obtain (1S,2R)-1-({[tert-butyl(diphenyl)silyl]oxy}methyl)-2-(hydroxymethyl)cyclopropyl 2-methoxyacetate, and its NMR data was the same as the title compound.

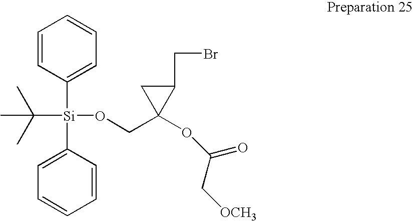

### Synthesis of (1R,2S)-1-({[tert-butyl(diphenyl)silyl]oxy}methyl)-2-(bromomethyl)cyclopropyl 2-methoxyacetate

The compound prepared in Preparation 24 (150 mg) was dissolved in 10 ml, of acetonitrile (AN), and 230 mg of triphenylphosphine (PPh₃) and 240 mg of carbontetrabromide (CBr₄) were slowly added dropwise thereto at 0° C. The resulting mixture was stirred for 1 hours at ambient temperature. Water (20 ml) and diethylether (50 ml) were added thereto. The organic layer therein was separated and removed by distillation under reduced pressure. The residue was purified by a silica gel column chromatography (eluent: ethylacetate/n-hexane: 1/8, v/v) to give 130 mg (yield 76%) of the title compound.

¹H NMR (CDCl₃) δ0.95 (t, 1H), 1.06 (s, 9H), 1.26 (dd, 1H), 1.77 (m, 1H), 3.25 (t, 1H), 3.39 (s, 3H), 3.74 (dd, 1H), 3.85 (q, 2H), 3.86 (d, 1H), 4.23 (d, 1H), 7.45 (m, 6H), 7.66 (m, 4 H).

¹³C NMR (CDCl₃) δ15.1, 15.8, 22.8, 26.4 (3C), 28.9, 55.9, 59.8, 62.6, 66.1, 124.4 (2C), 124.5 (2C), 126.5 (2C), 129.5, 129.6, 132.2 (4C), 166.2.

ESI: 492 (M+1)⁺, C24H31BrO4Si.

Furthermore, the same procedure as the above was conducted by using (1S,2R)-1-({[tert-butyl(diphenyl)silyl]oxy}methyl)-2-(hydroxymethyl)cyclopropyl 2-methoxyacetate as a starting material to obtain (1S,2R)-1-({[tert-butyl(diphenyl)silyl]oxy}methyl)-2-(bromomethyl)cyclopropyl 2-methoxyacetate, and its NMR data was the same as the title compound.

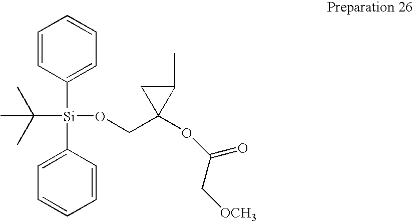

### Synthesis of (1R,2R)-1-({[tert-butyl(diphenyl)silyl]oxy}methyl)methylcyclopropyl 2-methoxyacetate

The compound prepared in Preparation 25 (120 mg) was dissolved in methanol (20 ml), and 20 mg of 10% Pd on Carbon was added thereto. The resulting mixture was reduced with hydrogen gas under 1 atm for 24 hours. The 10% Pd on Carbon (50 mg) was further added, and the resulting mixture was additionally reduced for 24 hours. The Pd on Carbon was removed by celite, the residual solution was removed by distillation under reduced pressure, and the residue was purified by a silica gel column chromatography (eluent: ethylacetate/n-hexane: 1/8, v/v) to give 80 mg (yield 79%) of the title compound.

¹H NMR (CDCl₃) δ0.53 (t, 1H), 1.01 (dd, 1H), 1.06 (s, 9H), 1.12 (d, 3H), 1.23 (m 1H), 3.42 (s, 3H), 3.83 (d, 1H), 3.89 (d, 2H), 4.14 (d, 1H), 7.41 (m, 6H), 7.65 (m, 4H).

ESI: 413 (M+1)+, C24H32O4Si.

Furthermnore, the same procedure as the above was conducted by using (1S,2R)-1-({[tert-butyl(diphenyl)silyl]oxy}methyl)-2-(bromomethyl)cyclopropyl 2-methoxyacetate as a starting material to obtain (1S,2S)-1-({[tert-butyl(diphenyl)silyl]oxy}methyl)methylcyclopropyl 2-methoxyacetate, and its NMR data was the same as the title compound.

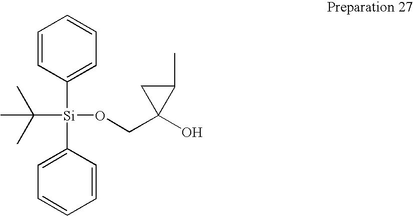

### Synthesis of (1R,2R)-1-({[tert-butyl(diphenyl)silyl]oxy}methyl)-2-methylcyclopropanol

The compound prepared in Preparation 26 (15 mg) was dissolved in 5 ml of ammonia dissolved in methyl alcohol (2M in MeOH), and the resulting mixture was stirred for 10 hours at ambient temperature. The solvent was removed by distillation under reduced pressure, and the residue was purified by a silica gel column chromatography (eluent: ethylacetate/n-hexane: 1/8, v/v) to give 12 mg (yield 98%) of the title compound.

¹H NMR (CDCl₃) δ0.06 (t, 1H), 0.88 (dd, 1H), 0.98 (d, 3H), 1.09 (s, 9H), 3.74 (dd, 1H), 3.87 (d, 1H), 7.42 (m, 6H), 7.71 (m, 4 H).

ESI: 341 (M+1)+, C21H28O2Si.

Furthermore, the same procedure as the above was conducted by using (1S,2S)-1-({[tert-butyl(diphenyl)silyl]oxy;}methyl)methylcyclopropyl 2-methoxy acetate as a starting material to obtain (1S,2S)-1-({[tert-butyl(diphenyl)silyl]oxy}methyl)-2-methylcyclopropanol, and its NMR data was the same as the title compound.

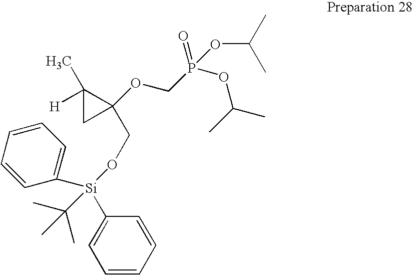

### Synthesis of diisopropyl {[(1R,2R)-1-({[tert-butyl(diphenyl)silyl]oxy}methyl)-2-methylcyclopropyl]oxy}methylphosphonate

The compound prepared in Preparation 27 (9 mg) was dissolved in 0.5 ml of dimethylformamide(DMF) solution in which 17.0 mg of diisopropyl bromomethylphosphonate was dissolved, and 5 mg of lithiumiodide (LiI) was added thereto. The lithium t-butoxide (LiOtBu) solution (0.11 ml) that 800 mg of lithium t-butoxide was dissolved in 10 ml of THF and 10 ml of DMF, was slowly added at 60-65° C. to the above solution in which the compound was dissolved. The resulting mixture was stirred for 10 hours at the same temperature. The solution was cooled to ambient temperature, and water (5 ml) and diethylether (50 ml) were added thereto. The organic layer therein was separated and removed by distillation under reduced pressure. The residue was purified by a silica gel column chromatography (eluent: ethylacetate/n-hexane: 1/4, v/v) to give 8 mg (yield 65%) of the title compound.

¹H NMR (CDCl₃) δ0.11 (t, 1H), 0.93 (m, 1H), 0.97 (d, 3H), 1.04 (s, 9H), 1.26 (d, 6H), 1.29 (d, 6H), 3.68 (d, 1H), 3.96 (d, 2H), 3.99 (d, 1H), 4.72 (m, 2H), 7.40 (m, 6H), 7.66 (m, 4 H).

ESI: 519 (M+1)+, C28H43O5PSi.

Furthermore, the same procedure as the above was conducted by using (1S,2S)-1-({[tert-butyl(diphenyl)silyl]oxy}methyl)-2-methylcyclopropanol as a starting material to obtain diisopropyl {[(1S,2S)-1-({[tert-butyl(diphenyl)silyl]oxy}methyl)-2-methylcyclopropyl]oxy}methylphosphonate, and its NMR data was the same as the title compound.

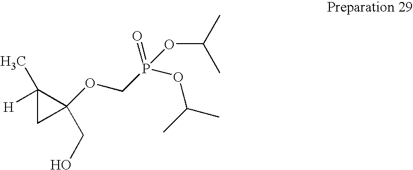

### Synthesis of diisopropyl {[(1R,2R)-1-(hydroxymethyl)-2-methylcyclopropyl]oxy}methylphosphonate

The compound prepared in Preparation 28 (7 mg) was dissolved in 1 ml of methyl alcohol, and 5 mg of ammonium fluoride (NH₄F) was added thereto. The resulting mixture was refluxed under heating for 4 hours. Alcohol was removed by distillation under reduced pressure, and the residue was purified by a silica gel column chromatography (eluent: methyl alcohol/dichloromethane: 5/95, v/v) to give 3 mg (yield 85%) of the title compound.

¹H NMR (CDCl₃) δ0.23 (t, 1H), 0.95 (m, 1H), 1.13 (d, 3H), 1.30 (d, 12H), 3.60 (d, 1H), 3.83 (d, 2H), 3.96 (d, 1H), 4.00 (s, 1H), 4.78 (m, 2H).

ESI: 281 (M+1)+, C12H25O5P.

The retention time of the compound thus obtained was measured after inducing according to the same procedure as the compound induced to measure the optical purity in Preparation 18. The value was 14 minutes (0.9 ml/min, Hexane:Isopropanol, 95:5), the same as that of the compound induced from the compound [the compound (13b) in Reaction Scheme 3]. Therefore, this compound has the same absolute configuration as the compound [[(−)-trans-isomer, the compound (13b) in Reaction Scheme 3] prepared in Preparation 18.

Furthermore, the same procedure as the above was conducted by using diisopropyl {[(1S,2S)-1-({[tert-butyl(diphenyl)silyl]oxy}methyl)-2-methylcyclopropyl]oxy}methylphosphonate as a starting material to obtain diisopropyl {[(1S,2S)-1-(hydroxymethyl)-2-methylcyclopropyl]oxy}methylphosphonate, and its NMR data was the same as the title compound. The retention time of the compound was measured after inducing according to the same procedure as the compound induced to measure the optical purity in Preparation 18. The value was 13 minutes (0.9 ml/min, Hexane:Isopropanol, 95:5), the same as that of the compound induced from the compound [the compound (13a) in Reaction Scheme 3]. Therefore, this compound has the same absolute configuration as the compound [[(+)-trans-isomer, the compound (13a) in Reaction Scheme 3] prepared in Preparation 18.

### EXAMPLE 1

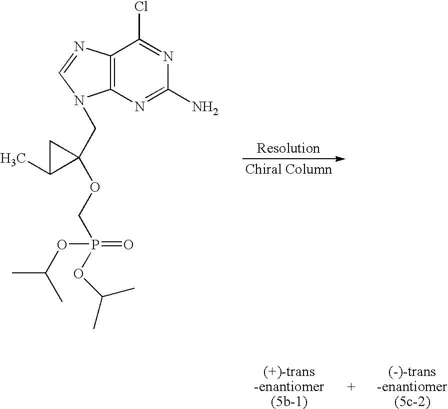

### Resolution of diisopropyl ({(±)-trans-1-[(2-amino-6-chloro-9H-purine-9-yl)methyl]-2-methylcyclopropyl}oxy)methylphosphonate

As described in the above Reaction Scheme 2, the racemate was resolved by a chiral column to obtain (+)-trans-optical isomer and (−)-trans-optical isomer. (±)-Trans-racemate (50 mg) obtained from Preparation 11 was passed through High performance liquid chromatography (eluent: hexane/isopropyl alcohol=80/20) fixed with a chiral column (Trade name: chiral pak AD, provided by DAICEL Chemical Industries, Ltd.) to obtain each 20 mg of (+)-trans-optical isomer, dilsopropyl ({(1S,2S)-1-[(2-amino-6-chloro-9H-purine-9-yl)methyl]-2-methylcyclopropyl}oxy)methylphosphonate (Compound 34) and (−)-trans-optical isomer and measure their optical activity (specific rotation). The optical isomer (5b-1) resolved in the front (Retention time: 7.8 minutes) was [α]_{D}=(+)16.35 (c=4.12 in CHCl₃), and the optical isomer (5c-1) resolved in the back (Retention time: 9.2 minutes) was [α]_{D}=(−)16.70 (c=1.92 in CHCl₃).

### EXAMPLE 2

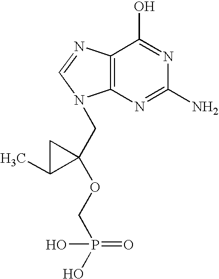

### Synthesis of (((1S,2S)-1-[(2-amino-6-hydroxy-9H-purine-9-yl)methyl]-2-methylcyclopropyl}oxy)methylphosphonic acid (Compound 1)

(+)-Trans-optical isomer (40 mg) resolved in Example 1 was dissolved in 8 ml of dichloromethane, and 285 mg of trimethylsilylbromide (TMSBr) was added thereto to reflux for 4 hours. Dichloromethane was distilled under reduced pressure to obtain a solid. The resulting solid was dissolved in 1N-HCl (10 ml) to reflux for 4 hours. After completing the reaction, water used as a solvent was distilled under reduced pressure, and the residue was solidified from methanol/ether (10/1) to obtain 25.4 mg (yield 83%) of the title compound as white solid.

[α]_{D}=(+)18.93 (c=0.66 in MeOH)

¹H NMR(MeOH-d4) δ0.71 (t, 1H), 1.13 (dd, 1H), 1.18 (d, 3H), 1.45 (m, 1H), 3.81 (dd, 1H), 3.98 (dd, 1H), 4.43 (d, 11H), 4.70 (d, 1H), 9.18 (s, 1H).

ESI: 330 (M+1), C11H16N5O5P

### EXAMPLE 3

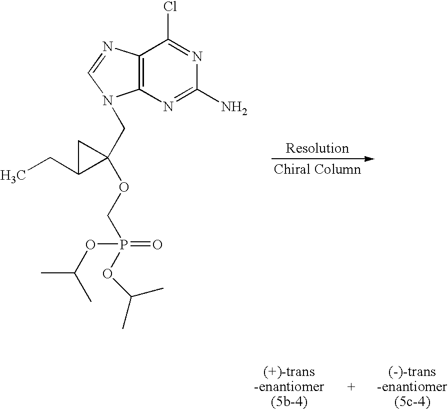

### Resolution of diisopropyl ({(±)-trans-1-[(2-amino-6-chloro-9H-purine-9-yl)methyl]-2-ethylcyclopropyl}oxy)methylphosphonate

As described in the above Reaction Scheme 2, racemates were resolved by a chiral column to obtain (+)-trans-optical isomer and (−)-trans-optical isomer. (±)-Trans-racemate (50 mg) obtained from Preparation 13 was passed through High Performance Liquid Chromatography (eluent: hexane/isopropyl alcohol=80/20) fixed with a chiral column (Trade name: chiral pak AD, provided by DAICEL Chemical Industries, Ltd.) to obtain each 20 mg of (+)-trans-optical isomer, diisopropyl ({(1S,2S)-1-[(2-amino-6-chloro-9H-purine-9-yl)methyl]-2-ethylcyclopropyl}oxy)methylphosphonate (Compound 35) (5b-4) and (−)-trans-optical isomer (5c-4) and measure their optical activity (specific rotation). The optical isomer resolved in the front (Retention time: 24 minutes) was [α]_{D}=(+)14.1 (c=7.37 in CHCl₃), and the optical isomer resolved in the back (Retention time: 27 minutes) was [α]_{D}=(−)14.2 (c=4.13 in CHCl₃).

### EXAMPLE 4

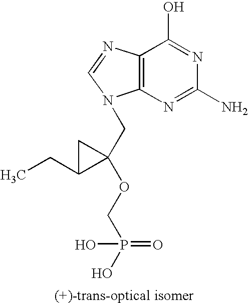

### Synthesis of ({(1S,2S)-1-[(2-amino-6-hydroxy-9H-purine-9-yl)methyl]-2-ethylcyclopropyl}oxy)methylphosphonic acid (Compound 12)

(+)-Trans-optical isomer (40 mg) resolved in Example 3 was reacted according to the same procedure as Example 2 to obtain 25.0 mg of the title compound as white solid.

[α]_{D}=(+)14.06 (c=0.32 in MeOH)

¹H NMR(MeOH-d4) δ0.76 (t, 1H), 1.03 (t, 3H), 1.10 (m, 1H), 1.38 (m, 1H), 1.47 (m, 2H), 3.80 (dd, 1H), 3.98 (dd, 1H), 4.33 (d, 1H), 4.75 (d, 1H), 9.20 (s, 1H).

ESI: 344 (M+1), C12H18N5O5P

### EXAMPLE 5

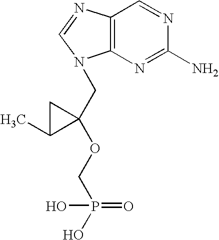

### Synthesis of ({(1S,2S)-1-[(2-amino-9H-purine-9-yl)methyl]-2-methylcyclopropyl}oxy)methylphosphonic acid (Compound 2)

(+)-Optical isomer (5b-1, 1.8 g) prepared in Example 1 was dissolved in 20 ml of methanol, 0.46 g of triethylamine (TEA) and 0.18 g of 10% Pd on C were added thereto, and the resulting mixture was reduced in hydrogen (1 atm) at 25° C. for 18 hours. The reactant was passed through cellite to remove Pd, and the obtained filtrate was distillated under reduced pressure to obtain the desired 6-dioxyguanidine derivative in 100% yield.

¹H NMR(CDCl₃) δ0.37 (t, 1H), 0.96 (m, 1H), 1.00 (d, 3H), 1.12(m, 1H), 1.14(m 12H), 3.79 (m, 2H), 21 (dd, 2H), 4.51 (m, 2H), 5.27 (brs, 2H), 8.01 (s, 1H).

The obtained 6-dioxyguanidine derivative (1.8 g) above was reacted according to the same procedure as Example 2 to obtain 1.3 g of the title compound (yield 100%).

¹H NMR(MeOH-d4) δ0.63 (t, 1H), 1.05 (dd, I H), 1.20 (d, 3H), 1.43 (m, 1H), 3.80 (m, 1H), 3.98 (m, 1H), 4.47 (d, 1H), 4.63 (d, 1H), 8.30 (s, 1H), 8.80 (s, 1H).

### EXAMPLE 6

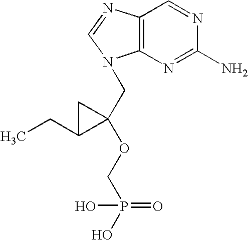

### Synthesis of ({(1S,²S)-1-[(2-amino-9H-purine-9-yl)methyl]-2-ethylcyclopropyl}oxy)methylphosphonic acid (Compound 13)

(+)-Optical isomer (5b-4, 400 mg) prepared in Example 3 was reacted according to the same procedure as Example 5 to obtain 270 mg of the title compound.

¹H NMR(MeOH-d4) δ0.71 (t, 1H), 1.10 (t, 3H), 1.12 (m, 1H), 1.37 (m, 1H), 1.50 (m, 2H), 3.80 (dd, 1H), 4.04 (dd, 1H), 4.26 (d, 1H), 4.74 (d, 1H), 8.68 (s, 1H).

### EXAMPLE 7

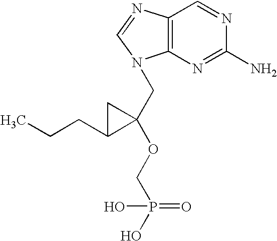

### Synthesis of ({(1S,2S)-1-[(2-amino-9H-purine-9-yl)methyl]-2-propylcyclopropyl}oxy)methylphosphon ic acid (Compound 24)

The compound (200 mg) prepared in Preparation 15 was reacted according to the same procedure as Example 5 to obtain 110 mg of ({(±)-trans-1-[(2-amino-9H-purine-9-yl)methyl]-2-propylcyclopropyl}oxy)methylphosphonic acid (Compound 39).

¹H NMR(MeOH-d4) δ0.71 (t, 1H), 0.96 (t, 3H), 1.10 (m, 1H), 1.43 (m, 3H), 1.47 (m, 2H), 3.78 (m, 1H), 4.01 (m, 1H), 4.26 (d, 1H), 4.71 (d, 1H), 8.68 (s, 1H), 8.74 (s, 1H).

Thereafter, the compound thus obtained was resolved according to the same procedure as Example 1 to obtain the title compound.

### EXAMPLE 8

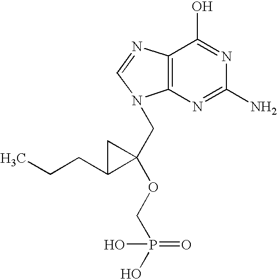

### Synthesis of ({(1S,2S)-1-[(2-amino-6-hydroxy-9H-purine-9-yl)methyl]-2-propylcyclopropyl}oxy)methylphosphonic acid (Compound 23)

The compound (150 mg) prepared in Preparation 15 was reacted according to the same procedure as Example 2 to obtain 110 mg of ({(±)-trans-1-[(2-amino-6-hydroxy-9H-purine-9-yl)methyl]-2-propylcyclopropyl}oxy)methylphosphonic acid (Compound 40).

1H NMR(MeOH-d4) δ0.74 (t, 1H), 0.96 (t, 3H), 1.11 (m, 1H), 1.42 (m, 5H), 3.79 (m, 1H), 3.96 (m, 1H), 4.32 (d, 1H), 4.75 (d, 1H), 9.17(s, 1H).

Thereafter, the compound thus obtained was resolved according to the same procedure as Example 1 to obtain the title compound.

### EXAMPLE 9

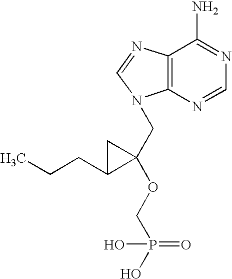

### Synthesis of (((1S,2S)-1-1(6-amino-9H-purine-9-yl)methyl]-2-propylcyclopropyl}oxy)methylphosphonic acid (Compound 26)

The compound (35 mg) prepared in Preparation 16 was dissolved in 10 ml of dichloromethane, and 280 mg of trimethylsilylbromide (TMSBr) was added thereto to reflux for 4 hours. Dichloromethane was distilled under reduced pressure to obtain a solid. The resulting solid was recrystallized in methanol/ether (10/1) to obtain 23 mg of ({(±)-trans-1-[(6-amino-9H-purine-9-yl)methyl]-2-propylcyclopropyl}oxy)methylphosphonic acid (Compound 41) as white solid.

¹H NMR(MeOH-d4) δ0.69 (t, 1H), 0.97 (t, 3H), 1.07 (m, 1H), 1.41 (m, 3H), 1.47 (m, 2H), 3.78 (m, 1H), 4.01 (m, 1H), 4.37 (d, 1H), 4.82 (d, 1H), 8.38 (s, 1H), 8.56 (s, 1H).

Thereafter, the compound thus obtained was resolved according to the same procedure as Example 1 to obtain the title compound.

### EXAMPLE 10

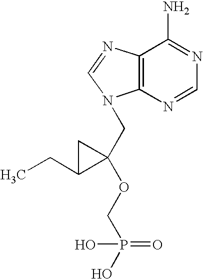

### Synthesis of ({(1S,2S)-1-[(6-amino-9H-purine-9-yl)methyl]-2-ethylcyclopropyl}oxy)methylphosphonic acid (Compound 14)

The compound (40 mg) prepared in Preparation 14 was reacted according to the same procedure as Example 9 to obtain 25 mg of ({(±)-trans-1-[(6-amino-9H-purine-9-yl)methyl]-2-ethylcyclopropyl}oxy) methylphosphonic acid (Compound 42).

¹H NMR(MeOH-d4) δ0.69 (t, 1H), 1.02 (t, 3H), 1.03 (m, 1H), 1.35 (m, 1H), 1.47 (m, 2H), 3.79 (m, 1H), 4.03 (m, 1H), 4.40 (d, 1H), 4.86 (d, 1H), 8.38 (s, 1H), 8.55 (s, 1H).

Thereafter, the compound thus obtained was resolved according to the same procedure as Example 1 to obtain the title compound.

### EXAMPLE 11

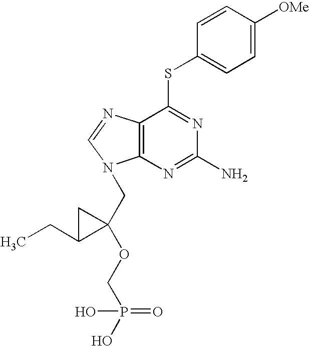

### Synthesis of [{(1S,2S)-1-({2-amino-6-1(4-methoxyphenyl)sulfanyl]-9H-purine-9-yl}methyl)-2-ethylcyclopropyl}oxy]methylphosphonic acid (Compound 15)

6-Chloroguanidine derivative (48mg) of the compound prepared in Preparation 13 was dissolved in 9 ml of ethanol, and 140 mg of triethylamine and 290 mg of 4-methoxythiocresole were added thereto. The resulting mixture was reacted under the reflux condition for 24 hours, and the reaction was completed by adding 20 ml of water. The reactant was distilled under reduced pressure to remove methanol, and the distilled reactant was extracted with dichloromethane and the extracting liquid was removed by distilling under reduced pressure. The residue was purified by a silica gel column to obtain the compound (40 mg), guanine of which 6-position was substituted by 4-methoxyphenylthio.

¹H NMR(CDCl₃) δ0.51 (t, 1H), 0.97 (t, 3H), 1.15 (m, 1H), 1.24(d, 6H), 1.27(d 6H), 1.40 (m, 3H), 3.80 (m, 2H), 3.80 (s, 3H), 4.12 (d, 1H), 4.37 (d, 1H), 4.68 (m, 2H), 4.78 (brs, 2H), 6.93 (m, 1H), 7.19 (m, 2H), 7.28 (m, 2H), 8.04 (s, 1H).

The resulting compound (40 mg) was reacted according to the same procedure as Example 9 to obtain 25 mg of [{(±)-trans-1-({2-amino-6-[(4-methoxyphenyl)sulfanyl]-9H-purine-9-yl}methyl)-2-ethylcyclopropyl }oxy]methylphosphonic acid (Compound 43).

¹H NMR(MeOH-d4) δ0.63 (t, 1H), 0.93 (t, 3H), 1.03 (m, 1H), 1.35 (m, 1H), 1.38 (m, 2H), 3.20 (m, 1H), 3.70 (m, 1H), 3.89 (m, 2H), 4.24 (m, 1H), 4.70 (m, 1H), 7.03 (d, 1H), 7.14 (m, 2H), 7.32 (m, 1H), 8.98 (s, 1H).

Thereafter, the compound thus obtained was resolved according to the same procedure as Example 1 to obtain the title compound.

### EXAMPLE 12

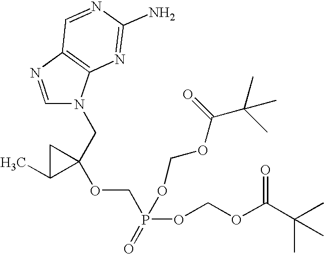

### Synthesis of (1S,2S)-3-[({1-[(2-amino-9H-purine-9-yl)methyl]-2-methylcyclopropyl}oxy)methyl]-8,8-dimethyl-3,7-dioxo-2,4,6-trioxa-3λ⁵-phosphanon-1-yl-pivalate (Compound 6)

The compound (600 mg) prepared in Example 5 was added to 5 ml of 1-methyl-2-pyrrolidinone. The mixture was heated to 60° C. and stirred for 30 minutes. To the resulting reactant, 0.58 g of triethylaniine and 0.86 g of chloromethylpivalate were added and the resulting mixture was stirred for 27 hours. The reactant was extracted with ethylacetate after lowering its temperature to 20° C. and completing the reaction by adding 20 ml of water. The reactant was distilled under reduced pressure to remove the extracting liquid. The residue was purified by a silica gel column chromatography to obtain 250 mg (yield 24%) of the title compound.

[α]_{D}=(+)20.57(c=2.04 in CHCl₃)

¹H NMR(CDCl₃) δ0.52 (t, 1H), 1.16 (m, 1H), 1.17 (d, 3H), 1.20(s, 18H), 1.41 (m 1H), 3.97 (m, 2H), 4.30 (q, 2H), 4.00 (brs, 2H), 5.64 (m, 4H), 8.05 (s, 1H), 8.69 (s, 1H).

### EXAMPLE 13

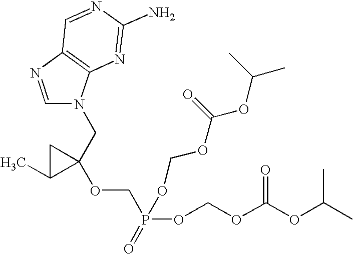

### Synthesis of (1S,2S)-bis {[(isopropoxycarbonyl)oxy]methyl}({1-[(2-amino-9H-purine-9-yl)methyl]-2-methylcyclopropyl}oxy)methylphosphonate (Compound 7)

The compound (0.98 g) prepared in Example 5 was added to 5 ml of 1-methyl-2-pyrrolidinone. The mixture was heated to 50° C. and stirred for 30 minutes. To the resulting reactant, 0.96 g of triethylamine and 1.44 g of chloromethylisopropylcarbonate were added and the resulting mixture was stirred for 3 hours. The reactant was extracted with ethylacetate after lowering its temperature to 20° C. and completing the reaction by adding 20 ml of water. The reactant was distilled under reduced pressure to remove the extracting liquid. The residue was purified by a silica gel column chromatography to obtain 270 mg (yield 16%) of the title compound.

[α]_{D}=(+)20.48(c=1.14 in CHCl₃)

¹H NMR(CDCl₃) δ0.49 (t, 1H), 1.15 (m, 1H), 1.16 (d, 3H), 1.29(m, 12H), 1.45 (m 1H), 3.97 (dd, 1H), 4.05 (dd, 1H), 4.30 (q, 2H), 4.90 (m, 2H), 4.62 (m, 4H), 8.05 (s, 1H), 8.69 (s, 1H).

### EXAMPLE 14

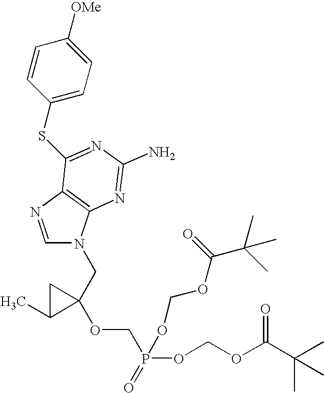

### Synthesis of (1S,2S)-3-{[(1-{[2-amino-6-(4-methoxyphenylthio)-9H-purine-9-yl]methyl}-2-methylcyclopropyl)oxy]methyl}-8,8-dimethyl-3,7-dioxo-2,4,6-trioxa-3λ⁵-phospanon-1-yl-pivalate (Compound 8)

6-Chloroguanidine derivative (48mg), the (+)-optical isomer compound prepared in Example 1, was dissolved in 9 ml of ethanol, and 140 mg of triethylamine and 290 mg of 4-methoxythiocresole were added thereto. The resulting mixture was reacted under the reflux condition for 24 hours, and the reaction was completed by adding 20 ml of water. The reactant was distilled under reduced pressure to remove methanol, and the distilled reactant was extracted with dichloromethane and the extracting liquid was removed by distilling under reduced pressure. The residue was purified by a silica gel column to obtain the compound, guanine of which 6-position was substituted by 4-methoxyphenylthio.

The resulting compound (40 mg) was reacted according to the same procedure as Example 9 to obtain phosphonic acid derivative (32 mg).

ESI: 452 (M+1)⁺C18H22N5O5PS

The above compound (30 mg) was reacted according to the same procedure as Example 13 to give 15 mg(yield 20%) of the title compound.

[α]_{D}=(+)13.75(c=2.36 in CHCl₃)

¹H NMR(CDCl₃) δ0.63 (t, 1H), 0.93 (t, 3H), 1.03 (m, 1H), 1.35 (m, 1H), 1.38 (m, 2H), 3.20 (m, 1H), 3.70 (m, 1H), 3.89 (m, 2H), 4.24 (m, 1H), 4.70 (m, 1H), 7.03 (d, 1H), 7.14 (m, 2H), 7.32 (m, 1H), 8.98 (s, 1H). 3) δ0.48 (t, 1H), 1.12 (m, 1H), 1.13 (d, 3H), 1.19(m, 18H), 1.38 (m 1H), 3.84 (s, 3H), 3.90 (dd, 1H), 3.98 (dd, 1H), 4.25 (q, 2H), 4.76 (brs, 2H), 5.62 (m, 4H), 6.95 (d, 2H), 7.54 (d, 2H), 7.91 (s, 1H).

### COMPARATIVE EXAMPLE 1

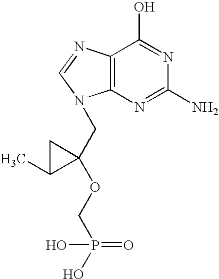

### Synthesis of {(−)-trans-1-[(2-amino-6-hydroxy-9H-purine-9-yl)methyl]-2-methylcyclopropyl}oxy)methylphosphonic acid (Compound 36)

(−)-Trans-optical isomer (40 mg) resolved in Example 1 was reacted according to the same procedure as Example 2 to obtain 20.1 mg (yield 80%) of the title compound as white solid.

[α]_{D}=(−)20.19 (c=1.21 in MeOH)

¹H NMR(MeOH-d4) δ0.71 (t, 1H), 1.13 (dd, 1H), 1.18 (d, 3H), 1.45 (m, 1H), 3.81 (dd, 1H), 3.98 (dd, 1H), 4.43 (d, 1H), 4.70 (d, 1H), 9.18 (s, 1H).

ESI: 330 (M+1), C11H16N5O5P

### COMPARATIVE EXAMPLE 2

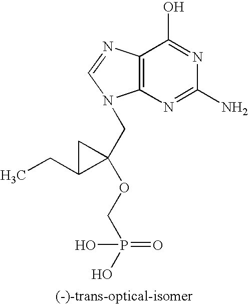

### Synthesis of ({(−)-trans-1-1(2-amino-6-hydroxy-9H-purine-9-yl)methyl]-2-ethylcyclopropyl}oxy)methylphosphonic acid (Compound 37)

(−)-Trans-optical isomer (40 mg) resolved in Example 3 was reacted according to the same procedure as Example 2 to obtain 20.0 mg of the title compound as white solid.

[α]_{D}=(−)13.47(c=1.47 in MeOH)

¹H NMR(MeOH-d4) δ0.76 (t, 1H), 1.03 (t, 3H), 1.10 (m, 1H), 1.38 (m, 1H), 1.47 (m, 2H), 3.80 (dd, 1H), 3.98 (dd, 1H), 4.33 (d, 1H), 4.75 (d, 1H), 9.20 (s, 1H).

ESI: 344 (M+1), C12H18N5O5P

### COMPARATIVE EXAMPLE 3

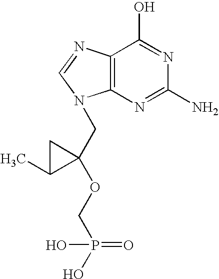

### Synthesis of ({(+)-cis-1-[(2-amino-6-hydroxy-9H-purine-9-yl)methyl]-2-methylcyclopropyl}oxy)methylphosphonic acid (Compound 38)

The compound (30 mg) prepared in Preparation 17 was reacted according to Example 2 to obtain 13 mg of the title compound.

¹H NMR(MeOH-d4) δ0.67 (t, 1H), 1.05 (dd, 1H), 1.13 (d, 3H), 1.38 (m, I1H), 3.90 (dd, 1H), 4.01 (dd, 1H), 4.22 (d, 1H), 4.58 (d, 1H), 9.17 (s, 1H).

ESI: 330 (M+1), C11H16N5O5P

### COMPARATIVE EXAMPLE 4

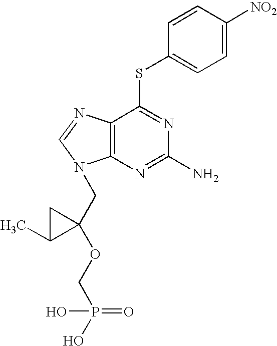

### Synthesis of [{(+)-cis-1-({2-amino-6-[(4-nitrophenyl)sulfanyl]-9H-purine-9-yl}methyl)-2-methylcyclopropyl}oxy]methylphosphonic acid (Compound 44)

6-Chloroguanidine derivative (48 mg), the compound prepared in Preparation 17, was dissolved in 9 ml of ethanol, and 140 mg of triethylamine and 290 mg of 4-nitrothiocresole were added thereto. The resulting mixture was reacted under the reflux condition for 24 hours, and the reaction was completed by adding 20 ml of water. The reactant was distilled under reduced pressure to remove methanol, and the distilled reactant was extracted with dichloromethane and the extracting liquid was removed by distilling under reduced pressure. The residue was purified by a silica gel column to obtain the compound (32 mg), guanine of which 6-position was substituted by 4-nitrophenylthio.

¹H -NMR(CDCl₃) δ0.62 (t, 1H), 0.93 (m, 1H), 1.16 (d, 3H), 1.26(d, 6H), 1.30(d, 6H), 1.36 (m, 1H), 3.79 (m, 1H), 3.92 (m, 1H), 3.98 (d, 1H), 4.38 (d, 1H), 4.74 (m, 2H), 4.83 (brs, 2H), 7.79 (d, 2H), 8.05 (s, 1H), 8.22 (d, 2H).

The resulting compound (32 mg) was reacted according to the same procedure as Example 9 to obtain 20 mg of the desired title compound.

¹H NMR(MeOH-d4) δ0.67 (t, 1H), 1.05 (m, 1H), 1.13 (t, 3H), 1.38 (m, 1H), 3.91 (m, 1H), 4.01 (m, 1H), 4.27 (m, 1H), 4.67 (m, 1H), 7.92 (d, 1H), 8.33 (m, 2H), 9.17 (s, 1H).

### COMPARATIVE EXAMPLE 5

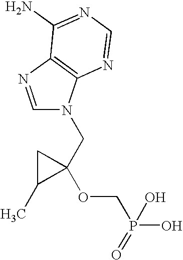

### Synthesis of ({[(+)-cis-[1-(6-amino-9H-purine-9-yl)methyl]-2-methylcyclopropyl}oxylmethylphosphonic acid (Compound 45)

The compound prepared in Preparation 12 (50 mg) was reacted according to the same procedure as Example 9 to obtain 40 mg of the title compound

¹H NMR(MeOH-d4) δ0.63 (t, 1H), 1.05 (m, 1H), 1I.10 (d, 3H), 1.32 (m, 1H), 3.87 (dd, 1H), 4.03 (dd, 1H), 4.28 (d, 1H), 4.71 (d, 1H), 8.37 (s, 1H), 8.50 (s, 1H).

The compound of the present invention exhibits a potent pharmacological effect to a hepatitis B cell line, HepG2.2.15, and a transgenic mouse, widely used for development of a therapeutic agent against hepatitis B, when intravenously or orally administered. The experimental procedures and results are described below.

Experiment 1

Measurement and Analysis of Inhibition Effect against Hepatitis B Virus (HBV)

(1) Cell Culture and Treatment with Drugs

HepG2.2.15 cell (M. A Shells et al., P.N.A.S. 84, 1005(1987)), a hepatocarcinoma cell line producing hepatitis B virus, was cultured in DMEM medium(GIBCO BRL, #430-2200) containing 10% FBS(Fetus bovine serum, GIBCO BRL, #16000-044), 1% ABAM (Antibiotic-Antimycotic, GIBCO BRL, #16000-028) and 400 μg/ml of geneticin(Sigma, #G-9516) in a T-75 flask under the conditions of 5% CO₂ incubator and 37° C. by dividing in a ratio of 1:3 at an interval of 3 days. The cells were distributed into a 96-well plate in the amount of 4×10⁴/well and then when 80-90% of cell density was achieved, the old medium was changed with 200 μl of DMEM medium containing 2% FBS, 1% ABAM and 400 μg/ml of geneticin. The drug solution was sequentially diluted five-fold each time, from 100 μM to 0.16 μM. In order to minimize an experimental error, each treatment was repeated 2-3 times for the respective drugs. The medium was changed every two days. On 10 days after the treatment with drug, 100 μl of the medium was collected and the degree of inhibition of viral replication by drugs was determined through quantitative PCR (Polymerase Chain Reaction).

(2) Determination of Cytotoxicity

After 100 μl of the medium was collected on 10th day from the treatment with drug, 7.5 mg/μl of MTT (Thiazolyl Blue Tetrazolium Bromide, Amresco, #0793-5G) solution was added to each well in the amount of 30 μl/well and each cell was cultured for 2 hours in a 5% CO₂ incubator at 37° C. The solution was discarded, and an isopropanol solution containing 10% Triton X-100 and 0.4 μl of c-HCl was added to each well in the amount of 120 μl/well. The cells thus dyed were transferred to the isopropanol solution by shaking for 2 hours. Absorbance at 540 nm was measured by Elisa Reader.

(3) PCR Estimation of Inhibition Effect on Hepatitis B Virus Replication

The degree of inhibition by drugs on the replication of hepatitis B virus was determined by using the cell culture solution collected on 10th day after the treatment with the drug. The cell culture solution treated with each drug was diluted ten-fold with distilled water and subjected to a pretreatment to destroy the cells by heating them for 15 minutes at 95° C. For the PCR amplification of the gene fragment of about 320 bp, the 2001-base position that is conserved in all sub-strain of hepatitis B virus and 2319-base position that is between the core antigen gene and polymerase gene were used as 5′-end and 3′-end primer, respectively. Then, the amount of genomic DNA of hepatitis B virus was quantified, and the inhibitory effect by drugs on the replication of hepatitis B virus was determined on the basis thereof.

First, the cell culture solution of hepatitis B virus that was not treated with drug was sequentially diluted and amplified through the PCR. The amplified DNA was subjected to electrophoresis on 2% agarose gel and stained with ethidium bromide (EtBr) to be analyzed by IS-1000 (Innotech Scientific Corporation) Digital Imaging System. Analysis of the cell culture solution treated with drug was then carried out by using the dilution fold in the range where linearity is maintained. The DNA obtained from the group treated with drug was amplified through the same PCR method, subjected to electrophoresis on 2% agarose gel, stained with ethidium bromide, and analyzed by IS-1000. The degree of inhibition by drugs in the viral replication was quantified by calculating the ratio of test group to control group. Table 2 summarizes the inhibitory effect (pharmaceutical activity and toxicity) of the typical compounds of the present invention.

TABLE 2

| Compound No. | EC₅₀ (μM) in HBV | CC₅₀ (μM) in HepG2.2.15 |
|---|---|---|
| 1 | 0.03 | >1000 |
| 2 | 1.0 | >1000 |
| 12 | 0.03 | >1000 |
| 13 | >10 | >1000 |
| 36 | >10.0 | >1000 |
| 37 | >40.0 | >1000 |
| 39 | >40.0 | >1000 |
| 40 | 1.2 | >1000 |
| 41 | >30.0 | >1000 |
| 42 | >30.0 | >1000 |
| 43 | >0.2 | >1000 |
| 38 | >10.0 | >1000 |
| 44 | >40.0 | >1000 |
| 45 | >40.0 | >1000 |

As can be seen from the results of Table 2, each of enantiomer and diasteroisomer has a high difference in pharmaceutical activity as an antiviral agent. Compounds 1 and 12, (+)-trans-optical isomer (enantiomer), among the above compounds exhibited the most excellent pharmaceutical activity.

Experiment 2

Pharmacological Test on Transgenic mouse (T/G mouse)

The compounds were administered via subcutaneous and oral routes in the following animal test.

The test compounds were administered to 4-5 week old HBV transgenic mice, which were obtained from FVB strain mice according to a method described in a reference (see, Jone D. Morrey, Kevin W. Bailey, Brent E. Korba, Robert W. Sidwell, Utilization of mice replicating high levels of hepatitis B virus for antiviral evaluation of lamivudine Antiviral research, 1999, 42, 97-108), subcutaneously for 9 days in the amount of 10 mg/kg/day and orally for 9 days in the amount of 10, 2 and 0.4 mg/kg/day, once a day, respectively (the same number of males and females were used). Blood was collected from the tail of the mouse and 5 tμl of serum was obtained during or after the administration of the drug. To this serum was added 15 ml of Genereleaser sol, which was then pretreated in different temperatures. HBV DNA was taken from the pretreated solution. The DNA was amplified by the PCR (Polymerase Chain Reaction) in the presence of 4 μl of 10×buffer (Perkin Elmer), 0.8 μl of 10 mM dNTP, 500 ng of the same HBV primers as used in Experiment 1, 2, 125 mM of MgCl₂, DMSO and Taq polymerase. The amount of HBV DNA was analyzed by electrophoresis in order to evaluate a pharmacological effect of the compound of the present invention. The results are described in the following Table 3. In the following Table 3, ^{┌}mice showing pharmacological effect_{┘} means the mice whose blood does not contain HBV DNA.

TABLE 3

| Compound No. | Amount(mg/kg/day) | Result* | Administration |
|---|---|---|---|
| 2 | 10 | 4/4 | subcutaneous |
| 6 | 1 | 5/5 | oral |
| 7 | 1 | 5/5 | oral |
| 8 | 1 | 2/5 | oral |

*The result means ┌ number of mice showing pharmacological effect / number of total mice ┘

As can be seen in the above Table 3, the compound of the present invention shows a potent hepatitis B therapeutic effect in the tested animals when orally or subcutaneously administered. Since Compounds 6 and 7 of the (+)-optical isomers exhibit very excellent pharmacological effect when they are orally administrated at 1 mpk or less, it is expected that the compounds of the present invention may be used very effectively for the treatment of hepatitis B.

1. (+)-Trans-isomers of (1-phosphonomethoxy-2-alkylcyclopropyl)methyl nucleoside derivatives represented by the following formula (1): 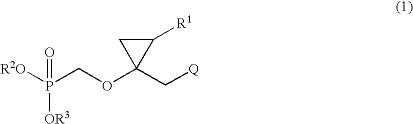
wherein,
R¹ represents C₁-C₇ alkyl,
R² and R³ independently of one another represent hydrogen, or represent C₁-C₄-alkyl optionally substituted by one or more substituents selected from a group consisting of halogen, C₁-C₄-alkoxy, phenoxy, C₇-C₁₀-phenylalkoxy, and C₂-C₅-acyloxy, or represent C₂-C₇-acyl, C₆-C₁₂-aryl, C₁-C₇-alkylaminocarbonyl, di(C₁-C₇-alkyl)aminocarbonyl or C₃-C₆-cycloalkylaminocarbonyl, or represent —(CH₂)ₘ—OC(═O)—R⁴ wherein m denotes an integer of 1 to 12 and R⁴represents C₁-C₁₂-alkyl, C₂-C₇-alkenyl, C₁-C₅-alkoxy, C₁-C₇-alkylamino, di(C₁-C₇-alkyl)amino, C₃-C₆-cycloalkyl, or 3- to 6-membered heterocycle having 1 or 2 hetero atoms selected from a group consisting of nitrogen and oxygen,
Q represents a group having the following formulae: 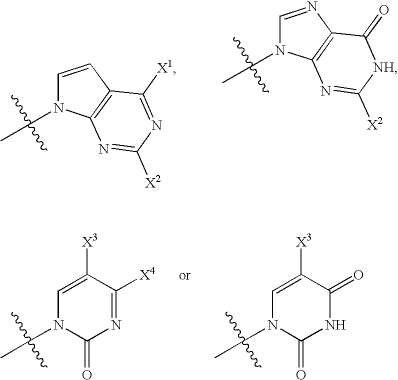
wherein,
X¹, X₂, X₃ and X⁴ independently of one another represent hydrogen, amino, hydroxy, or halogen, or represent C₁-C₇-alkyl, C₁-C₅-alkoxy, allyl, hydroxy-C₁-C₇-alkyl, phenyl, or phenoxy, each of which is optionally substituted by nitro or C₁-C₅-alkoxy, or represent C₆-C₁₀-arylthio which is optionally substituted by nitro, amino, C₁-C₆-alkyl, or C₁-C₄-alkoxy, or represent C₆-C₁₂-arylamino, C₁-C₇-alkylamino, di(C₁-C₇-alkyl)amino, C₃-C₆-cycloalkylamino, or a structure of 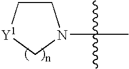
wherein n denotes an integer of 1 or 2 and Y₁ represents O, CH₂, or N—R (R represents C₁-C₇-alkyl or C₆-C₁₂-aryl), pharmaceutically acceptable salts, hydrates or solvates thereof.

2. The compounds of claim 1 wherein the pharmaceutically acceptable salt is salt with sulfuric acid, methanesulfonic acid or hydrohalic acid.

3. The compounds of claim 1 wherein
R₁ represents C₁-C₃ alkyl,
R² and R³ independently of one another represent hydrogen, or represent C₁-C₄-alkyl optionally substituted by one or more substituents selected from a group consisting of fluorine, C₁-C₄-alkoxy, and phenoxy, or represent —(CH₂)ₘ—OC(═O)—R⁴ wherein m denotes an integer of 1 to 12, and R⁴ represents C₁-C₅-alkyl or C₁-C₅-alkoxy,
Q represents 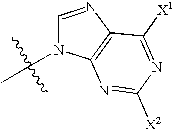
wherein, X¹ represents hydrogen, hydroxy, amino or 4-methoxyphenylthio, or 4-nitrophenylthio, and X² represents hydrogen or amino.

4. The compounds of claim 1 which are selected from the group consisting of the compounds described in the following Tables 1a and 1b:
TABLE 1a

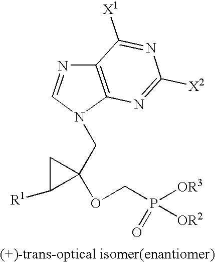

| COM. NO. | R¹ | R² & R³ | X¹ | X² |
|---|---|---|---|---|
| 1 | CH₃ | H | OH | NH₂ |
| 2 | CH₃ | H | H | NH₂ |
| 3 | CH₃ | H | NH₂ | H |
| 4 | CH₃ | H | 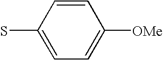 | NH₂ |
| 5 | CH₃ | H | Cl | NH₂ |
| 6 | CH₃ | 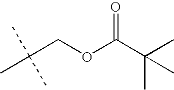 | H | NH₂ |
| 7 | CH₃ | 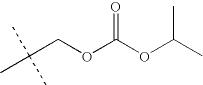 | H | NH₂ |
| 8 | CH₃ | 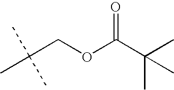 | 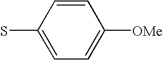 | NH₂ |
| 9 | CH₃ | 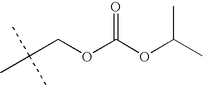 | 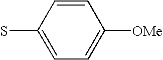 | NH₂ |
| 10 | CH₃ | 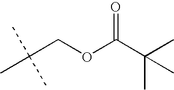 | NH₂ | H |
| 11 | CH₃ | 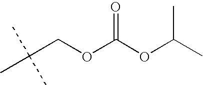 | NH₂ | H |
| 12 | C₂H₅ | H | OH | NH₂ |
| 13 | C₂H₅ | H | H | NH₂ |
| 14 | C₂H₅ | H | NH₂ | H |
| 15 | C₂H₅ | H | 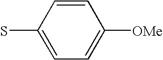 | NH₂ |

TABLE 1b

| | | | | |
|---|---|---|---|---|
| 16 | C₂H₅ | H | Cl | NH₂ |
| 17 | C₂H₅ | 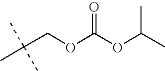 | H | NH₂ |
| 18 | C₂H₅ | 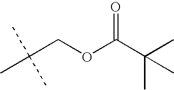 | H | NH₂ |
| 19 | C₂H₅ | 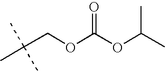 | NH₂ | H |
| 20 | C₂H₅ | 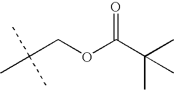 | NH₂ | H |
| 21 | C₂H₅ | 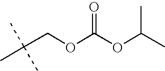 | 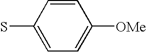 | NH₂ |
| 22 | C₂H₅ | 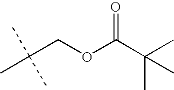 | 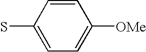 | NH₂ |
| 23 | C₃H₇ | H | OH | NH₂ |
| 24 | C₃H₇ | H | H | NH₂ |
| 25 | C₃H₇ | H | Cl | NH₂ |
| 26 | C₃H₇ | H | NH₂ | H |
| 27 | C₃H₇ | H | 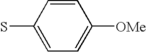 | NH₂ |
| 28 | C₃H₇ | 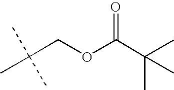 | H | NH₂ |
| 29 | C₃H₇ | 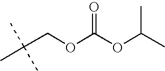 | H | NH₂ |
| 30 | C₃H₇ | 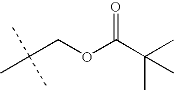 | NH₂ | H |
| 31 | C₃H₇ | 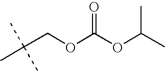 | NH₂ | H |
| 32 | C₃H₇ | 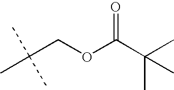 | 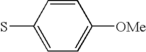 | H |
| 33 | C₃H₇ | 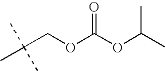 | 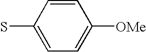 | H |
| 34 | CH₃ | iso-propyl | Cl | NH₂ |
| 35 | C₂H₅ | iso-propyl | Cl | NH₂ |

5. A process for preparing a compound represented by the following formula (2): 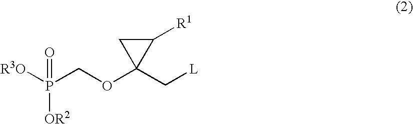
in which R¹, R² and R³ are defined as in claim 1, and L represents methanesulfonyloxy, p-toluenesulfonyloxy, or halogen, characterized in that
(a) an ethylglycolate, the alcohol group of which is protected, as represented by the following formula (6): 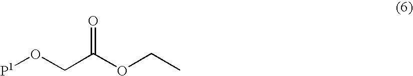
in which P¹ represents an alcohol-protecting group selected from a group consisting of benzyl(Bn), tetrahydropiranyl(THP), t-butydiphenylsilyl(TBDPS) and t-butyldimethylsilyl(TBDMS), is reacted with alkyl magnesium halide represented by the following formula (7):
R⁷—MgX (7)
in which R⁷ represents C₃-C₇ alkyl and X represents halogen, in the presence of titanium tetraisopropoxide[Ti(OiPr)₄],
(b) the resulting two cyclopropanol diastereoisomers represented by the following formulae (8) and (9): 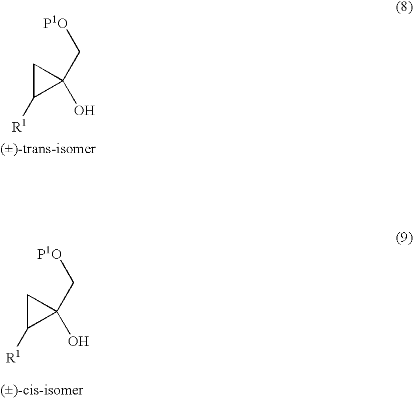
in which R¹ is defined as in claim 1 and P¹ is defined as previously described, are resolved with a silica gel column,
(c) each compound resolved in the step (b) is subjected to an ether-forming reaction with a compound represented by the following formula (10): 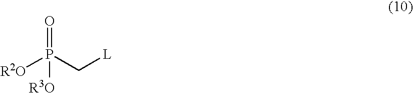
in which R² and R³ are defined as in claim 1, and L is defined previously described, in the presence of base to produce a phosphonate compound represented by the following formula (11) or (12): 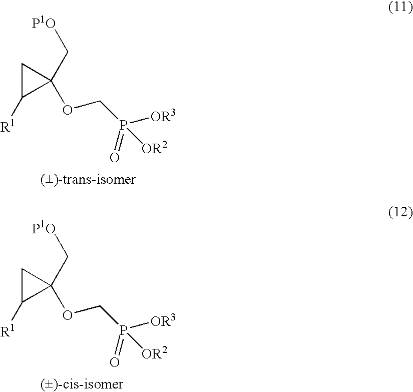
in which R¹, R² and R³ are defined as in claim 1, and P¹ is defined as previously described, and
(d) an alcohol-protecting group of the resulting compound of formula (11) or (12) is removed and a leaving group (L) is introduced to produce a compound represented by the following formula (2a) or (2b): 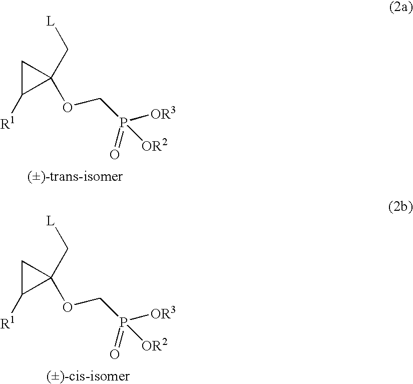
in which R¹, R² and R³ are defined as in claim 1, and L is defined as previously described.

6. A compound represented by the following formula (8): 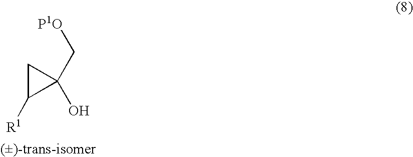
in which R¹ is defined as in claim 1, and P¹ represents an alcohol-protecting group selected from a group consisting of benzyl(Bn), tetrahydropiranyl(THP), t-butydiphenylsilyl(TBDPS) and t-butyldimethylsilyl(TBDMS), and stereoisomers thereof.

7. A process for preparing stereoisomer of the compound of formula (1) as defined in claim 1 characterized in that a compound represented by the following formula (4a) or (4b): 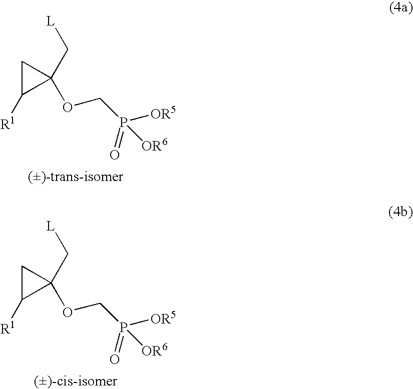
in which R¹ is defined as in claim 1, L represents methanesulfonyloxy, p-toluenesulfonyloxy, or halogen, and R⁵ and R⁶ independently of one another represent C₁-C₇-alkyl, is reacted with a compound represented by the following formula (3):
QH (3)
in which Q is defined as in claim 1, and each compound thus obtained is resolved with a chiral column or chiral reagents to produce (+), (−) two optical isomers, each of which is present as an enantiomer enriched isomer, and then each of them is treated with trimethylsilylbromide(TMSBr) to produce the corresponding (+), (−) two optical isomers of a compound represented by the following formula (1a): 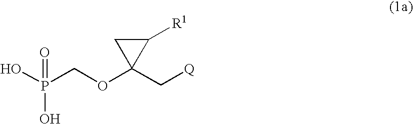
in which R¹ and Q are defined as in claim 1, and if necessary, groups R^{2′}- and R^{3′} are introduced into the compound thus obtained to produce the corresponding optical isomers of a compound represented by the following formula (1b): 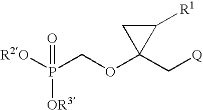
in which R¹ and Q are defined as in claim 1, and R^{2′} and R^{3′} represent R² and R³ with the exception of hydrogen, respectively.

8. A process for preparing stereoisomer of the compound of formula (1) as defined in claim 1 characterized in that a compound represented by the following formula (13) or (14): 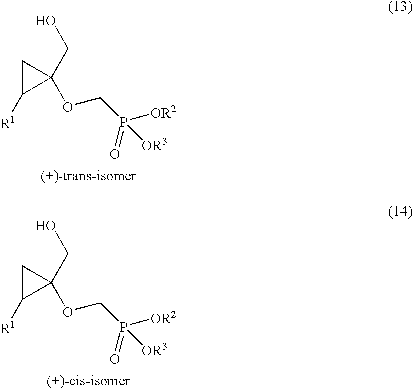
in which R¹, R² and R³ are defined as in claim 1, that is obtained by removing an alcohol-protecting group in a compound represented by the following formula (11) or (12): 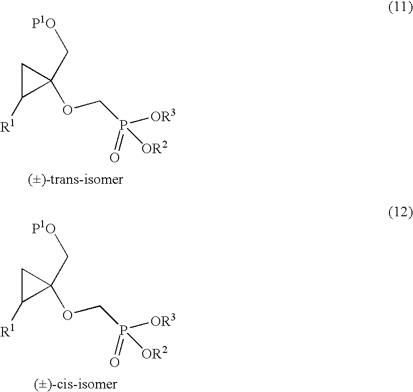
in which R¹, R² and R³ are defined as in claim 1, and P¹ represents an alcohol-protecting group selected from a group consisting of benzyl(Bn), tetrahydropiranyl(THP), t-butydiphenylsilyl(TBDPS) and t-butyldimethylsilyl(TBDMS), is resolved with a hydrolase (lipase) to produce enantiomer enriched compounds represented by the following formulae (13a) and (13b) or (14a) or (14b): 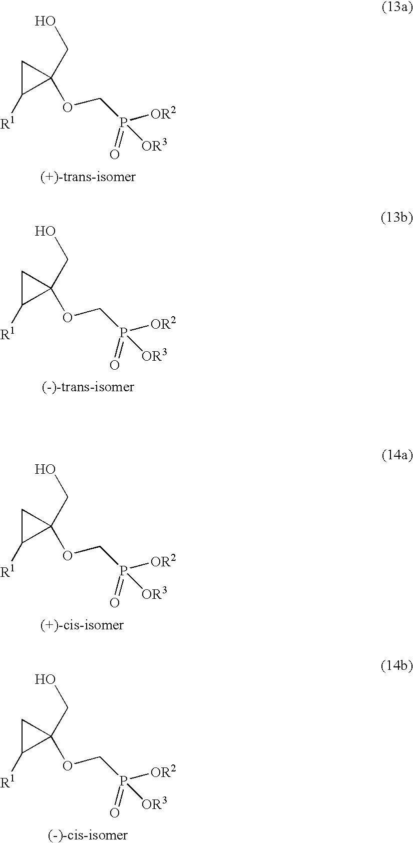
in which R¹, R² and R³ are defined as in claim 1, and further an alcohol group in the compound of formula (13 a), (13b), (14a) or (14b) thus obtained is replaced with a leaving group (L) to produce a compound represented by the formula (2aa), (2ab), (2ba) or (2bb): 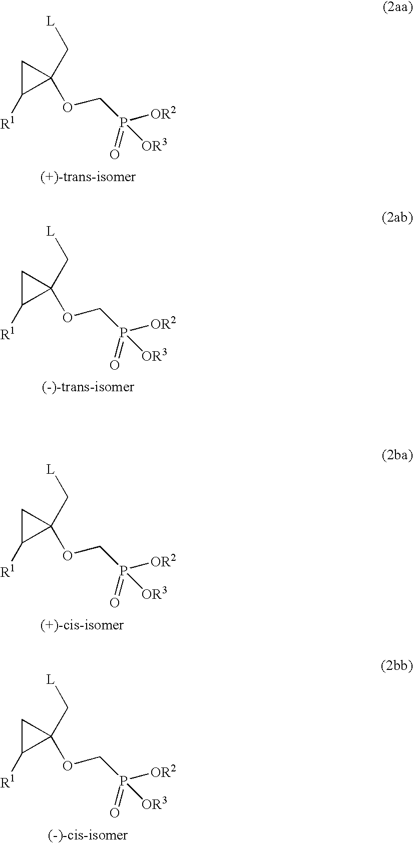
in which R¹, R² and R³ are defined as in claim 1, and L represents methanesulfonyloxy, p-toluenesulfonyloxy, or halogen, and the resulting compound is reacted with a compound represented by the formula (3):
QH (3)
in which Q is defined as in claim 1, to produce the enantiomer enriched compound of formula (1).

9. A process for preparing stereoisomer of the compound of formula (1) as defined in claim 1 characterized in that
aa) an alcohol-protecting group (P²) is introduced into (+)-(methylenecyclopropyl)carbinol or (−)-(methylenecyclopropyl)carbinol, whose absolute configuration is known,
bb) the resulting compound is subjected to dihydroxylation reaction,
cc) an alcohol-protecting group (P¹) is introduced into the primary hydroxy group in the compound obtained in the above bb) step and an alcohol-protecting group (P³) is introduced into the tertiary hyroxy group to produce a compound represented by the formula (15a), (15b), (16a) or (16b): 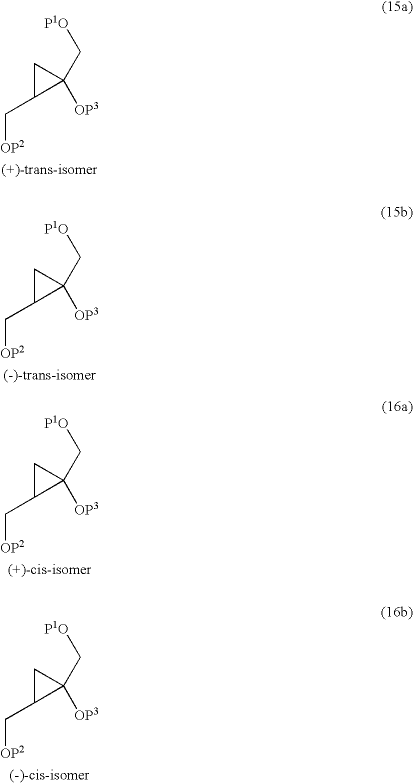
in which P¹ represents an alcohol-protecting group selected from a group consisting of benzyl(Bn), tetrahydropiranyl(THP), t-butydiphenylsilyl(TBDPS) and t-butyldimethylsilyl(TBDMS), P² represents benzyl, benzoyl, 4-methoxybenzyl, methyloxybenzyl, methyloxymethyl or trityl and P³ represents 1-methoxyacetyl, acetyl or 2-(trimethylsilyl)-1-ethanesulfony,
dd) the protecting group (P²) in the resulting compound is removed selectively, the leaving group (L) is introduced, and the compound thus obtained is subjected to a reduction reaction or substituted with C₁-C₇-alkyl group,
ee) the protecting group (P³) in the compound thus obtained in the above dd) step is removed to produce a compound represented by the following formula (8a), (8b), (9a) or (9b): 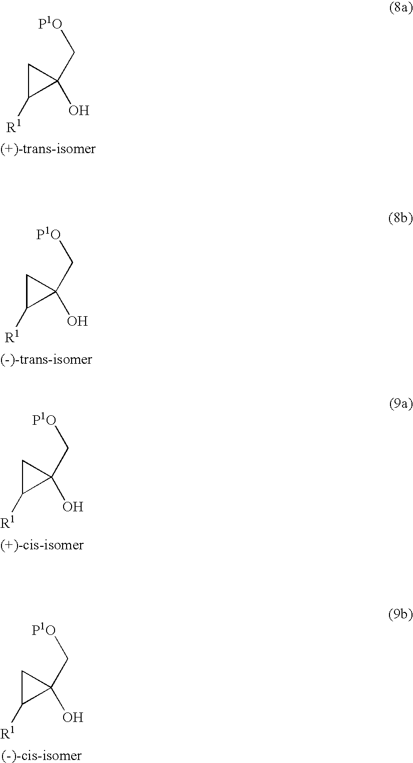
in which R¹ is defined as in claim 1, and P¹ represents an alcohol-protecting group selected from a group consisting of benzyl(Bn), tetrahydropiranyl(THP), t-butydiphenylsilyl(TBDPS) and t-butyldimethylsilyl(TBDMS),
ff) the resulting compound in the above step ee) is reacted with a phosphonate compound represented by the following formula (10): 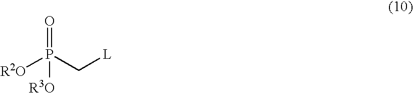
in which R² and R³ are defined as in claim 1, and L represents methanesulfonyloxy, p-toluenesulfonyloxy, or halogen, and the protecting group (P¹) of the compound thus obtained is removed to produce a compound represented by the following formula (13a), (13b), (14a) or (14b): 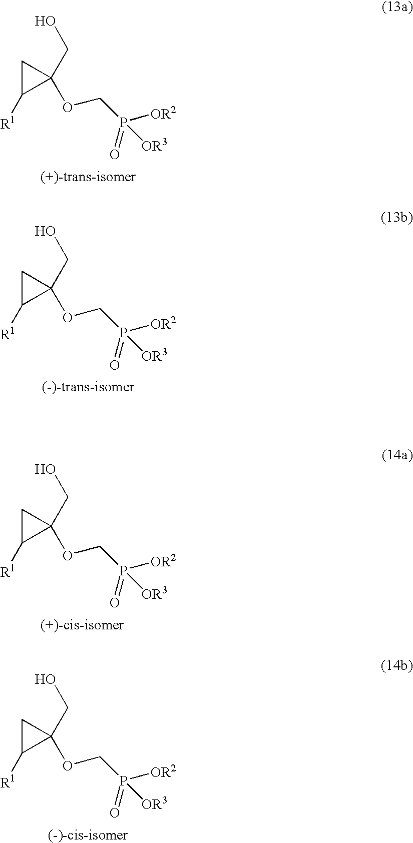
in which R¹, R² and R³ are defined as in claim 1,
gg) an alcohol group of the resulting compound is replaced with the leaving group (L) to produce a compound represented by the following formula (2aa), (2ab), (2ba) or (2bb): 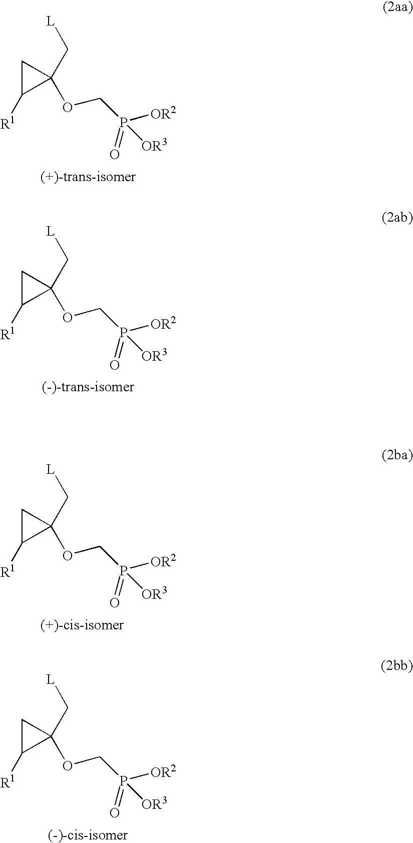
in which R¹, R² and R³ are defined as in claim 1, and L represents methanesulfonyloxy, p-toluenesulfonyloxy, or halogen, and
hh) the resulting compound is reacted with a compound represented by the following formula (3):
QH (3)
in which Q is defined as in claim 1, to produce the enantiomer enriched compound of formula (I).

10. A composition for the treatment of viral diseases, which comprises as an active ingredient (+)-trans-isomer of (1-phosphonomethoxy-2-alkylcyclopropyl)methyl nucleoside derivative of formula (1) as defined in claim 1, pharmaceutically acceptable salt, hydrate, or solvate thereof together with the pharmaceutically acceptable carrier.

11. A composition for the treatment of hepatitis B, which comprises as an active ingredient (+)-trans-isomer of (1-phosphonomethoxy-2-alkylcyclopropyl)methyl nucleoside derivative of formula (1) as defined in claim 1, pharmaceutically acceptable salt, hydrate, or solvate thereof together with the pharmaceutically acceptable carrier.
